(19) United States
(12) Patent Application Publication
(10) Pub. No.: US 2026/0162793 A1
(43) Pub. Date: Jun. 11, 2026

(54) OPHTHALMIC HEALTH MANAGEMENT SYSTEM

(71) Applicant: ALCON INC., Fribourg (CH)

(72) Inventors: Evan Brecht-Curry, Seattle, WA (US); Paul Nolan Houdashell, Grand Prairie, TX (US); Aaron Davidson, Cincinnati, OH (US); Viraj Shah, Duluth, GA (US); Travis Beaven, Burleson, TX (US); Kobe Lee Alexander Ryan, Woodinville, WA (US); Diana Renee Milam, Woodway, TX (US); Abraham Cole Lawless, Arlington, TX (US); Jamie McNeil, Conshohocken, PA (US)

(21) Appl. No.: 19/409,226

(22) Filed: Dec. 4, 2025

Related U.S. Application Data
(60) Provisional application No. 63/728,934, filed on Dec. 6, 2024.
(60) Provisional application No. 63/899,060, filed on Oct. 14, 2025.

(51) Int. Cl. G16H 20/00; G16H 50/20

(52) U.S. Cl. CPC G16H 20/00; G16H 50/20

(57) ABSTRACT

A method of ophthalmic health management may include obtaining health data corresponding to a user. Environmental data corresponding to a location associated with the user may be obtained. Based on the health data and the environmental data, a symptom of the user may be determined. An action may be determined in response to determining the symptom. A notification may be provided to the user via a device associated with the user based on the determined action.

### CROSS-REFERENCE TO RELATED APPLICATION

This application claims priority to U.S. Provisional Patent Application No. 63/899,060, filed on Oct. 14, 2025 and U.S. Provisional Patent Application No. 63/728,934, filed Dec. 6, 2024, the disclosures of each of which are incorporated herein by reference in their entireties.

### FIELD

The present disclosure relates to a system configured to help manage ophthalmic health.

### BACKGROUND

Eye irritants (e.g., allergens—such as pollen, particles—such as smoke, dust pollutants, etc.) in the environment may vary depending on numerous factors, including geographical location, seasonal changes, weather patterns, wind conditions, population density, natural disasters (e.g., wildfires, volcanic eruptions, etc.) and/or local flora. Additionally, lifestyle factors may contribute to eye irritation. For example, prolonged screen time (e.g., blue light exposure), extended reading sessions, inadequate sleep, swimming (e.g., chlorine exposure), and/or other lifestyle decisions may cause and/or exacerbate eye irritation. Potential treatments for ophthalmic irritation may include a wide range of options, from over-the-counter eye drops to prescription medications and/or lifestyle modifications.

### SUMMARY

The present disclosure relates to operations that include obtaining health data corresponding to a user. The operations may further include obtaining environmental data corresponding to a location associated with the user. The operations may further include determining, based on the health data and the environmental data, a symptom. The operations may further include determining an action in response to determining the symptom. The operations may further include providing a notification to the user via a device associated with the user based on the determined action.

### BRIEF DESCRIPTION OF THE DRAWINGS

The present disclosure relates to systems and methods for ophthalmic health management, wherein:

FIG. 1A illustrates an example environment in which an ophthalmic health management system may be implemented, according to one or more embodiments of the present disclosure;

FIG. 1B illustrates an example patient side of the example environment of FIG. 1A, according to one or more embodiments of the present disclosure;

FIG. 1C illustrates an example provider side of the example environment of FIG. 1A, according to one or more embodiments of the present disclosure;

FIG. 2 illustrates an example process 200 that may be performed to manage ophthalmic health in response to allergens, according to one or more embodiments of the present disclosure;

FIG. 3 is a flow diagram illustrating a method of ophthalmic health management, according to one or more embodiments of the present disclosure; and

FIG. 4 is a block diagram of an example computing system suitable for use in implementing one or more embodiments of the present disclosure.

### DETAILED DESCRIPTION

The management of eye conditions related to environmental and/or lifestyle factors presents unique challenges in the healthcare landscape. Eye allergies, in particular, often result in significant discomfort and reduced quality of life for patients. Current allergen tracking systems typically provide general information about pollen and other allergen levels without specifically addressing the implications for eye health or offering personalized recommendations for ophthalmic care. Patient behaviors also may negatively impact ophthalmic health without their knowledge. For example, patients may be unaware that their prolonged screen exposure may be contributing to their symptoms. This gap in contextual information and/or lack of behavioral awareness may leave patients uncertain about the relationship between their symptoms and environmental and/or behavioral triggers, potentially leading to inappropriate self-treatment or delayed care.

For example, the correlation between environmental allergen levels and ophthalmic conditions represents a complex relationship that may not be well understood by many individuals and/or that may vary significantly from person to person. The impact of allergens on eye health may be particularly complex, as different individuals may react differently to various allergens. Some people may experience mild irritation, while others may suffer from more severe symptoms. Furthermore, the connection between environmental allergens and eye health may not be immediately apparent to many individuals, potentially leading to delayed or inadequate treatment. These variations may make it challenging for individuals to be aware of the specific irritants that may affect them at any given time. Additionally, people may not always be cognizant of the full range of irritants that may trigger eye irritation, as some allergens may be less common or less well-known.

Moreover, the effectiveness of various treatments may vary depending on the specific allergens present and the individual's unique physiological response. This variability may further complicate the process of selecting and implementing appropriate treatments for eye allergy symptoms. Individuals may also not be fully aware of all the available treatment options or may struggle to determine which treatments are most appropriate for their specific situation. This lack of awareness may result in suboptimal management of eye allergy symptoms and reduced quality of life for affected individuals.

In addition, traditional allergen monitoring services typically provide broad geographic data without specifically addressing the implications for ophthalmic health. This disconnect may lead to suboptimal management of eye conditions, as patients may fail to recognize the relationship between elevated allergen levels and their ophthalmic symptoms.

For instance, eye discomfort resulting from allergen exposure may manifest in various ways, including redness, itching, tearing, burning, and/or general irritation. These symptoms may overlap with other ophthalmic conditions such as dry eye syndrome, potentially leading to misidentification of the underlying cause by patients. For example, when patients experience eye discomfort, they may attribute symptoms to various causes, such as dry eye syndrome, when the actual culprit may be an allergic reaction to environmental allergens. Without proper guidance, patients may select products designed for different conditions such that the selected products may fail to address the specific allergen-related mechanisms causing their discomfort—thus resulting in suboptimal symptom relief.

Furthermore, the episodic nature of traditional eye care delivery models, which typically involve annual examinations, may not adequately address the dynamic and seasonal nature of allergen-related eye conditions. Pollen counts, air quality, and other environmental factors may fluctuate significantly throughout the year and across different geographic locations. These variations may necessitate adjustments in treatment approaches that may not be anticipated during scheduled appointments.

The integration of real-time environmental data with patient-specific ophthalmic information may enable more timely and appropriate interventions. By combining allergen forecasts with individual patient profiles, healthcare systems may better serve patients through personalized notifications that may prompt proactive management of eye conditions. Such systems may bridge the gap between periodic doctor visits by providing ongoing guidance based on changing environmental conditions and/or individual susceptibility.

Additionally, patients may lack awareness about the specific products designed to address different eye conditions, or they may not recognize when environmental conditions warrant the use of such products. Moreover, patient compliance with recommended treatments for eye conditions may be compromised by lack of awareness regarding when environmental conditions warrant intervention. Often individuals may seek treatment only after experiencing significant discomfort, rather than taking preventative measures when allergen levels first begin to rise. This reactive approach may result in prolonged periods of unnecessary discomfort and potential complications.

The fragmentation between environmental monitoring systems and healthcare delivery platforms may create barriers to effective information exchange. Patients may need to independently research allergen forecasts and then determine the appropriate ophthalmic response without professional guidance. This disconnected process may lead to delays in appropriate care and suboptimal management of symptoms.

Geographic mobility also presents additional challenges, as patients may travel between regions with varying allergen profiles. Without location-aware monitoring and notification systems, individuals may be unprepared for changes in environmental conditions that may affect their eye health. This lack of preparedness may be particularly problematic for contact lens wearers, who may experience heightened sensitivity to environmental allergens.

The relationship between eye care professionals and patients typically revolves around discrete office visits, with limited opportunities for ongoing communication regarding changing environmental conditions. This communication gap may result in missed opportunities for timely intervention and adjustments to treatment plans based on current allergen levels.

Traditional product recommendation mechanisms therefore often fail to incorporate real-time environmental data, personal health data, and/or location-specific factors when suggesting ophthalmic products. This lack of personalization may result in generic recommendations that do not address the specific needs of individual patients based on their current circumstances and/or environmental exposures.

In addition, educational resources regarding the relationship between environmental allergens and eye health may not be readily accessible to patients at the moments when such information would be most relevant. The absence of contextual education may contribute to knowledge gaps that affect patients'ability to make informed decisions about their eye care, particularly during periods of elevated allergen levels.

Patients may also lack awareness regarding how their daily behaviors and habits may negatively impact their ophthalmic health. This knowledge gap may lead to inadvertent exacerbation of eye conditions and symptoms. For instance, patients may not recognize that wearing contact lenses during high allergen periods without appropriate precautions may potentially increase their allergen exposure. Additionally, patients may be unaware that improper lens cleaning or replacement schedules may compound issues related to environmental irritants. An ophthalmic health management system that correlates geographic allergen data and/or patient behavior data with ophthalmic health data may therefore improve patient education, enhance compliance with appropriate treatments, and/or strengthen the continuity of care between patients and their eye care professionals.

The integration of behavioral awareness into an ophthalmic health management system may provide patients with a more comprehensive understanding of factors affecting their eye health. By combining information about environmental allergen levels with personalized guidance regarding behavioral modifications, such systems may offer more effective support for patients managing their symptoms. This approach may bridge the gap between environmental monitoring and behavioral awareness, potentially leading to improved symptom management.

Furthermore, patients may be unaware that other behaviors not directly associated with allergens may also negatively impact their ophthalmic health. For example, patients may spend significant amounts of time viewing screens (e.g., phone screens) without recognizing the potential correlation between their screen time and their eye discomfort. The extended use of digital devices may contribute to reduced blink rates, which may in turn affect tear film distribution and potentially lead to dry eye symptoms. However, patients may not connect these behaviors with their discomfort, potentially attributing their symptoms to environmental factors and/or preexisting ophthalmic conditions.

Improving patient education regarding these behavioral factors may improve comprehensive ophthalmic care as a result. By increasing awareness of how daily behaviors may impact eye health, patients may be able to make more informed decisions about their activities. This enhanced understanding may enable patients to modify their behaviors proactively, potentially reducing the severity of symptoms and improving overall ophthalmic health outcomes.

Furthermore, an ophthalmic health care management system that shifts from a reactive model of ophthalmic care delivery to a proactive model of ophthalmic care delivery may improve patient outcomes by reducing symptoms and/or potentially preventing symptoms before onset. For example, an ophthalmic health care management system that identifies, selects, and/or orders ophthalmic products for patients based on patient-specific data may allow a patient to address ophthalmic symptoms before onset and/or before symptoms are exacerbated. For instance, an ophthalmic health care management system that may identify, select, and/or order ophthalmic products based on allergen data and/or user-behavior data corresponding to a patient. Thus, products may be selected and ordered for patients before they know they need to use the products. As a result, symptoms due to allergies and/or user-behaviors may be prevented before onset and/or may be mitigated in severity.

The present disclosure relates to an ophthalmic health management system that may determine a person's symptoms (e.g., currently existing and/or future symptoms) based on their current and/or future presence in an environment. For example, the ophthalmic health management system may determine potential ophthalmic symptoms based on the allergen profile in their current and/or future location. The ophthalmic health management system may generate customized actions with respect to the determined symptoms. The ophthalmic health management system may include a computing system configured to collect and process data from multiple sources to generate personalized risk notifications for users based on their symptoms. In some embodiments, these notifications may be specifically tailored to address ophthalmic health of the user based on geographic allergen profiles, user health data, and/or user location data.

The ophthalmic health management system may be designed to improve patient awareness regarding the manner in which their behaviors and/or allergens in their locations may impact their ophthalmic health. Thus, the ophthalmic health management system may be configured to improve prevention and/or treatment of eye allergy symptoms through personalized notifications. The notifications may include various types of information and recommendations tailored to the user's specific situation. For example, the notifications may provide real-time updates on local pollen counts, air quality indices, and/or other environmental events that may impact eye health. The ophthalmic health management system may also send reminders to use prescribed eye drops or other medications associated with the user, to use lens care solutions to clean their contact lenses, and/or to perform other user actions when allergen levels exceed a threshold in the user's area. Furthermore, the notifications may suggest preventive measures such as wearing sunglasses, avoiding outdoor activities during peak allergy hours, and/or reducing screentime, among other preventative measures.

The ophthalmic health management system may provide product recommendations based on the user's specific ophthalmic symptoms and/or symptom severity. For instance, in response to a user frequently experiencing itchy eyes during high pollen seasons, the notification may suggest purchasing antihistamine eye drops when pollen count is high. The recommendations may be further personalized based on the user's purchase history and preferences (e.g., brand preferences).

The ophthalmic health management system may generate a personalized allergen avoidance plan for the user, suggesting specific times of day to limit outdoor exposure based on local allergen forecasts. Notifications may provide tips on proper eye hygiene practices, such as washing hands frequently and avoiding touching or rubbing the eyes. The ophthalmic health management system may offer guidance on how to create an allergen-free environment at home, including recommendations for air purifiers or hypoallergenic bedding. Users may also receive reminders to schedule regular check-ups with an eye care professional, especially during allergy seasons.

Information on alternative treatments, such as cold compresses or artificial tears, may be provided to help alleviate eye allergy symptoms. The ophthalmic health management system may also provide guidance on proper contact lens care and usage during allergen events to minimize eye irritation.

The ophthalmic health management system may keep users informed about the latest research and treatments for eye allergies, providing information on new options for managing their symptoms. Reports on the user's symptom patterns and treatment effectiveness over time may be generated, which may be shared with their healthcare provider to inform treatment decisions.

Based on the allergen analysis, the ophthalmic health management system may initiate purchasing actions for individual patients. For example, when pollen levels exceed a threshold in a user's location, the system may generate orders and/or information to be used in orders for ophthalmic products, and/or place orders for ophthalmic products such as allergy eye drops or other recommended products. As a result, the patients may have ready access to ophthalmic products before their symptoms manifest.

The ophthalmic health management system may also be configured to monitor a patient's eyes via a software application that may be included on patient devices. This software application may utilize the device's camera to monitor the patient's eyes for certain symptoms or conditions associated with allergies, user behaviors, and/or other ophthalmic issues. The camera-based monitoring system may provide real-time data on various ophthalmic characteristics that may help identify symptoms and track treatment effectiveness. The ophthalmic health management system may capture and analyze multiple characteristics of the eye through the device camera. For example, the ophthalmic health management system may monitor the redness of the eyes by analyzing the blood vessel patterns visible in the sclera (white part of the eye).

The ophthalmic health management system may utilize pattern recognition to identify specific types of allergic reactions. For instance, seasonal allergic conjunctivitis may present differently from perennial allergic conjunctivitis or contact lens-related allergies. By recognizing these patterns, the ophthalmic health management system may suggest more targeted treatments.

The ophthalmic health management system may track changes in symptoms over time in relation to treatment use. For example, the ophthalmic health management system may document reductions in redness or swelling following the use of antihistamine eye drops, providing objective evidence of treatment efficacy. This tracking feature may help patients and healthcare providers assess whether a particular treatment is working effectively.

Based on the monitored eye characteristics, the ophthalmic health management system may generate condition assessments that may suggest potential diagnoses. For example, a combination of redness, tearing, and eyelid swelling coinciding with high pollen counts may suggest seasonal allergic conjunctivitis. The ophthalmic health management system may present these assessments along with confidence levels based on the strength of the symptom pattern.

The ophthalmic health management system may use machine learning algorithms to improve symptom determination accuracy over time. The ophthalmic health management system may incorporate user-reported symptoms alongside camera-based observations. For instance, users may report itching or burning sensations that may not be directly observed but may be used for condition assessment when combined with image data.

The ophthalmic health management system may feature a telemedicine component that may allow users to share eye monitoring data directly with their eye care professionals. This capability may enable remote assessment of eye conditions and may reduce the need for in-person visits for minor issues or follow-up assessments.

For contact lens wearers, the ophthalmic health management system may provide specialized monitoring to detect signs of contact lens-related allergies or complications. The ophthalmic health management system may track changes in eye characteristics that may occur after lens insertion or removal, helping to identify whether symptoms may be related to lens wear or environmental allergens.

The ophthalmic health management system may be configured to provide personalized alerts based on detected eye conditions. For example, in response to the ophthalmic health management system detecting an increase in eye redness and eye swelling during high pollen periods, the system may generate a notification suggesting the use of allergy eye drops or temporary discontinuation of contact lens wear.

Through continuous monitoring of eye characteristics and correlation with treatment usage, the ophthalmic health management system may help enhance treatment regimens. The ophthalmic health management system may identify which treatments may be effective for a particular user's symptoms and may suggest timing for medication administration based on symptom patterns.

The ophthalmic health management system may also track compliance with prescribed treatments by analyzing changes in eye characteristics following scheduled medication times. Consequently, the ophthalmic health management system may help healthcare providers understand whether lack of improvement may be due to treatment ineffectiveness or poor adherence.

The eye monitoring capabilities of the ophthalmic health management system may thus allow patients and/or healthcare providers to manage ophthalmic conditions (e.g., eye conditions resulting from allergens) and improve treatment approaches based on objective, quantifiable data collected in real-time environments.

In some embodiments, the ophthalmic health management system may operate within the context of digital health and e-commerce platforms, specifically within the ophthalmic care ecosystem. Additionally or alternatively, the ophthalmic health management system may interface with mobile devices and applications used by patients, allergen data sources, electronic health record systems including patient ophthalmic data, e-commerce platforms for eye care products, and/or cloud-based data processing and storage systems.

The embodiments of the present disclosure may be utilized with any suitable system, apparatus, or device in which health management may be beneficial. For example, in some embodiments, an ophthalmic health management system may be configured to perform operations pertaining to the ophthalmic health of a patient based on environmental data such as allergen data and health data of the patient. For instance, the ophthalmic health management system may determine symptoms (e.g., future symptoms and/or existing symptoms) based on the allergen data and the health data, may determine an action (e.g., preventative, therapeutic, curative, educative, palliative, and/or diagnostic actions, among others) to take in response to the determination of the symptoms, and may provide a notification to the patient based on the determined action. The action determined by the symptom may include generating a diagnosis, selecting one or more products associated with the symptom, generating an order for one or more products associated with the symptom, and/or generating a treatment plan based on the symptom, among other actions. By notifying patients of potential symptoms that they may experience due to allergens in their location, the ophthalmic health management system may improve patient care and patient outcomes and/or may enhance patient awareness that allergen levels may be correlated with their ophthalmic health.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to an ophthalmic health management system in relation to allergens. However, such uses are not meant to be limiting such that the ophthalmic health management system described may be used in any number of different contexts and applications where it may be helpful or applicable. Additionally, while described with respect to the management of ophthalmic health, it will be appreciated that the system may be implemented in other health areas in which the environment may impact a patient's health.

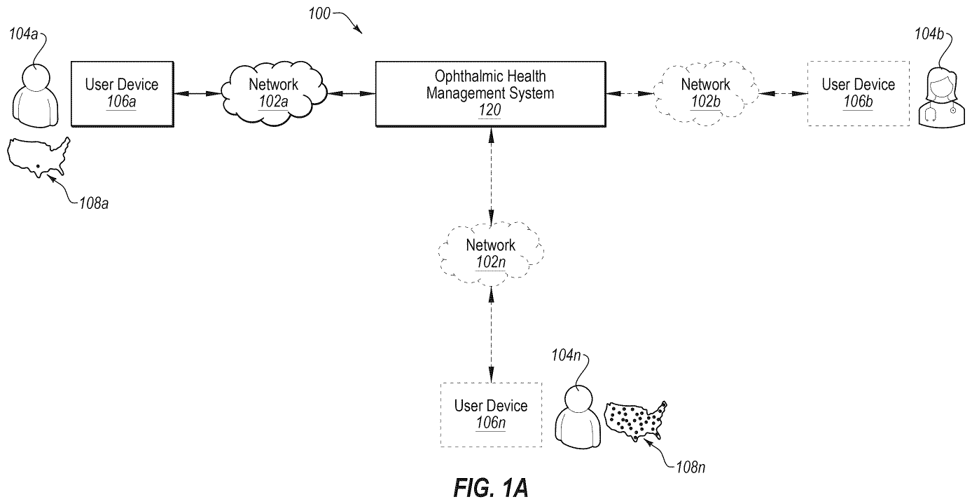

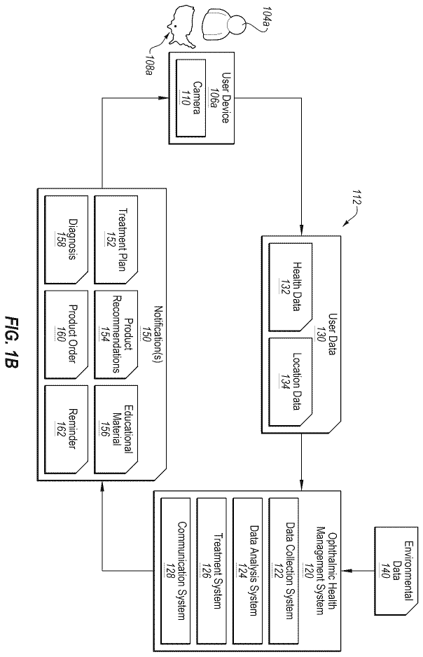

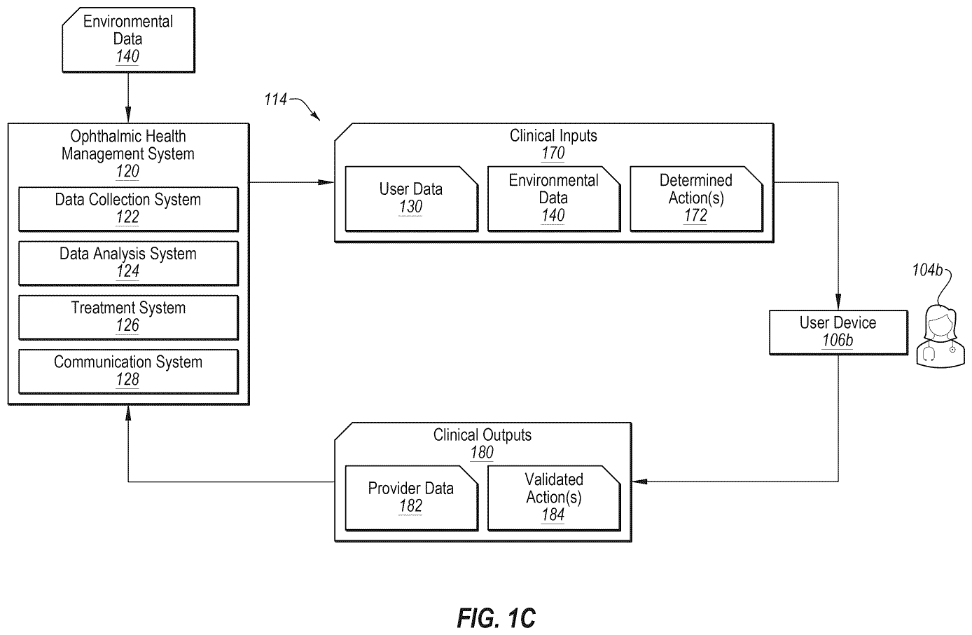

FIG. 1A illustrates an example environment 100 in which an ophthalmic health management system 120 may be implemented. The example environment 100 may include one or more networks 102a-102n (collectively, “the networks 102”), one or more user devices 106a-106n (collectively, “the user devices 106”), and the ophthalmic health management system 120.

In some embodiments, a first network 102a may be configured to communicatively couple a first user device 106a and the ophthalmic health management system 120. In some embodiments, a second network 102b may be configured to communicatively couple a second user device 106b and the ophthalmic health management system 120. In some embodiments, additional networks 102n may be configured to communicatively couple additional user devices 106n and the ophthalmic health management system 120.

In some embodiments, the networks 102 may include any network or configuration of networks configured to send and receive communications between devices and/or systems. In some embodiments, the networks 102 may include a conventional type of network, a wired or wireless network, and may have numerous different configurations. Furthermore, the networks 102 may include a local area network (LAN), a wide area network (WAN) (e.g., the Internet), or other interconnected data paths across which multiple devices and/or entities may communicate.

In some embodiments, the networks 102 may include a peer-to-peer network and/or a client-server network. The networks 102 may also be coupled to or may include portions of a telecommunications network for sending data in a variety of different communication protocols. In some embodiments, the networks 102 may include Bluetooth® communication networks or cellular communication networks for sending and receiving communications and/or data. The networks 102 may also include a mobile data network that may include fourth-generation (4G), fifth-generation (5G), long-term evolution (LTE), long-term evolution advanced (LTE-A), Voice-over-LTE (“VoLTE”) or any other mobile data network or combination of mobile data networks. Further, the networks 102 may include one or more IEEE 802.11 wireless networks. In some embodiments, the networks may share various portions of one or more networks. For example, the networks 102 may include the Internet or some other network.

The user devices 106 may be any electronic or digital device. For example, the first user device 106a, the second user device 106b, and/or the additional user devices 106n may include or may be included in a desktop computer, a laptop computer, a smartphone, a mobile phone, a tablet computer, a smart television, a wearable device such as smart glasses, or any other electronic device with a processor that is configured to enable user communication. In some embodiments, the user devices 106 may each include computer-readable-instructions stored on one or more computer-readable media that are configured to be executed by one or more processors to perform operations described in this disclosure.

In some embodiments, the first user device 106a may be associated with the first user 104a. In some embodiments, the first user device 106a may be associated with the first user 104a based on the first user 104a being the owner of the first user device 106a and/or the first user device 106a being controlled by the first user 104a. For example, the first user device 106a may be controlled by the first user 104a when the first user device 106a is obtaining commands and/or input from the first user 104a. For instance, the first user device 106a may obtain input from the first user 104a via a user interface included in the first user device 106a. As another example, the first user device 106a may be controlled by the first user 104a when the first user 104a is logged into a user account on the first user device 106a. For instance, the first user 104a may be logged into a user account associated with the first user 104a and the ophthalmic health management system 120.

In some embodiments, the first user 104a may be a patient. As used in the present disclosure, the term “patient” may include any individual who may receive information related to their personal health via the ophthalmic health management system 120 and/or who may have products selected and/or identified for them by the ophthalmic health management system 120 based on patient health data and/or environmental data corresponding to the patient.

In some embodiments, the first user 104a may be associated with a location 108a. In some embodiments, the location 108a may include the current location of the first user device 106a. For example, the first user device 106a may utilize global positioning, Wi-Fi positioning, cell tower triangulation, Bluetooth beaconing, sensor-based location estimation, IP address geolocation, and/or other location estimating techniques to determine the current location of the first user device 106a. In these and other embodiments, the current location of the first user device 106a may be associated with the first user 104a. In some embodiments, the location 108a may include a future location of the first user 104a and/or past location of the first user 104a. For example, the location 108a may be determined based on data obtained from the first user device 106a indicating a future location of the first user 104a.

In some embodiments, the location 108a (e.g., current, future, and/or past location) may be determined based on application data (e.g., data from mobile travel applications such as the United Airlines® mobile application), calendar data, commuting pattern data, location history data, email data, messaging data, notification data, and/or other data that may be obtained from the first user device 106a that may correspond to the first user 104a. For example, data obtained from the first user device 107a may indicate the location 108a of the first user 104a. For instance, a current, future, and/or past location of the first user 104a may be determined based on data from the first user device 106a corresponding to a hotel reservation, a restaurant reservation, a car rental reservation, a plane ticket, a train ticket, an event ticket (e.g., a ticket to a concert, sporting event, or other events), an appointment, a meeting, and/or other data that may indicate the location 108a of the first user 104a. In these and other embodiments, the location 108a may be a current location, a future location, and/or a past location. For example, the location 108a may be a future location determined based on an airline reservation associated with the first user 104a.

In some embodiments, the first user 104a may be associated with multiple locations 108. For example, the first user 104a may be associated with a current location and a future location and/or multiple future locations. As another example, the first user 104a may be associated with a current location, a future location, and/or a past location.

In some embodiments, the second user device 106b may be associated with a second user 104b. In some embodiments, the second user device 106b may be associated with the second user 104b based on the second user 104b being the owner of the second user device 106b and/or the second user device 106b being controlled by the second user 104b. For example, the second user device 106b may be controlled by the second user 104b when the second user device 106b is obtaining commands and/or input from the second user 104b. For instance, the second user device 106b may obtain input from the second user 104b via a user interface included in the second user device 106b. As another example, the second user device 106b may be controlled by the second user 104b when the second user 104b is logged into a user account on the second user device 106b. For instance, the second user 104b may be logged into a user account associated with the second user 104b and the ophthalmic health management system 120.

In some embodiments and as illustrated in FIGS. 1A and 1C, the second user 104b may be a healthcare provider. As used in the present disclosure, the term “healthcare provider” may include any individual who may receive information related to the personal health of the first user 104a and/or the additional users 104n and generate clinical outputs (e.g., medical consultation, diagnosis, treatment, and/or advice, among other clinical outputs) based on the personal health information of the first user 104a and/or the additional users 104n.

In some embodiments, the additional user devices 106n may each be associated with the additional users 104n. In some embodiments, each additional user device 106n may be associated with a respective additional user 104n in a similar manner as the first user device 106a is associated with the first user 104a and the second user device 106b is associated with the second user 106n. In some embodiments, each of the additional users 104n may be patients. In these and other embodiments, the additional users 104n may each be associated with an additional location 108n, which may be determined in a similar manner as described previously with respect to the location 108a. In some embodiments, the additional locations 108n associated with the additional users 104n may be the same and/or different than the location 108a associated with the first user 104a. For example, as shown in FIG. 1A, the additional users 104n may be distributed in various locations throughout the United States. In these and other embodiments, the additional locations 108n may be current locations, future locations, and/or past locations of the additional users 104n.

In some embodiments, each of the user devices 106 may be configured to send data to and receive data from the ophthalmic health management systems 120 via a respective network 102. For example, the first user device 106a may provide user data corresponding to the first user 104a to the ophthalmic health management system 120, and the additional user devices 106n may provide user data corresponding to the additional users 104n to the ophthalmic health management system 120. For instance, as described in more detail with respect to FIG. 1B, the first user device 106a may provide health data (e.g., ophthalmic health data) associated with the first user 104a and/or location data associated with the location 108a to the ophthalmic health management system 120, and may receive notifications based on the health data and/or location data from the ophthalmic health management system 120 (e.g., including treatment plans, diagnoses, product recommendations, product orders, educational materials, and/or reminders).

As another example, as described in more detail with respect to FIG. 1C, the second user device 106b may receive clinical inputs from the ophthalmic health management system 120, and may provide clinical outputs to the ophthalmic health management system 120. For instance, as described in more detail with reference to FIG. 1C, the ophthalmic health management system 120 may provide user data corresponding to the first user 104a, environmental data corresponding to the location 108a associated with the first user 104a, and/or suggested actions to the second user device 106b, and the second user device 106b may provide health data and/or validated actions to the ophthalmic health management system 120.

In some embodiments, the ophthalmic health management system 120 may include any suitable system, apparatus, or device that may be configured perform health management with respect to a user 104. For example, in some embodiments, the ophthalmic health management system 120 may include code and routines configured to allow a computing system to perform one or more ophthalmic management operations. Additionally or alternatively, the ophthalmic health management system 120 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the ophthalmic health management system 120 may be implemented using a combination of hardware and software.

In some embodiments, at least a portion of the ophthalmic health management system 120 may be integrated or included in any or all of the user devices 106. In some embodiments, the ophthalmic health management system 120 may be at least partially integrated and/or included in one or more digital and/or e-commerce platforms (e.g., the MARLÖ® digital eye care platform from Alcon®).

In some embodiments, an ophthalmic health management module may be included on each of the user devices 106 that may direct operations associated with ophthalmic health management with respect to the user devices 106, and/or may enable the user devices 106 to interact with the ophthalmic health management system 120. In some embodiments, the ophthalmic health management module may be included in the ophthalmic health management system 120. In these and other embodiments, the ophthalmic health management module may be implemented as a software module, a hardware module, or a combination of software and hardware modules. For example, the ophthalmic health management module may be a software application included on the user devices 106 that may direct the user device to perform one or more operations associated with ophthalmic health management. For instance, the ophthalmic health management module may obtain the user data 130 and/or control the camera 110 of the user device 106a as described with respect to FIG. 1B. In these and other embodiments, operations described as being performed by the user devices 106 throughout this disclosure may be performed by an ophthalmic health management module stored on the user devices 106 and/or a separate device.

Generally, the ophthalmic health management system 120 may be configured to analyze data obtained from a user device 106 and/or other sources to determine a symptom of a user (e.g., a patient) 104, determine an action in response to determining the symptom of the user 104, and/or provide a notification to the user 104 based on the determined action. For example, the ophthalmic health management system 120 may be configured to obtain and analyze allergen data, user health data (e.g., ophthalmic health data including user behavior data that may impact ophthalmic health), and user location data to determine a particular symptom associated with allergens in a location 108 associated with a user 104. In these and other embodiments, the ophthalmic health management system 120 may determine a current symptom and/or a future symptom associated with the user 104. In these and other embodiments, the ophthalmic health management system 120 may be configured to perform one or more operations associated with mitigating and/or preventing ophthalmic symptoms based on the data obtained by the ophthalmic health management system 120.

In some embodiments, the ophthalmic health management system 120 may access various sources to obtain data related to ophthalmic health management. In some embodiments, the sources accessed by the ophthalmic health management system 120 may include Electronic Health Record (EHR) systems, Electronic Medical Record (EMR) systems, webpages, databases, and/or other sources that may provide data related to ophthalmic health management. For example, the ophthalmic health management system 120 may obtain data from webpages through the use of application programming interfaces (APIs), data scraping tools, and/or other data collection tools. For instance, the ophthalmic health management system 120 may access allergen monitoring websites such as Pollen.com® to obtain allergen data. In some embodiments, the ophthalmic health management system 120 may be configured to process the data obtained from the user devices 106 and/or other sources to generate personalized notifications for the users 104 and/or to order products for the users 104.

In these and other embodiments, the ophthalmic health management system 120 may obtain data based on the locations 108 of the users 104 in the environment 100. For example, the ophthalmic health management system 120 may obtain allergen data corresponding to the locations 108 associated with each user 104 in the environment 100. For instance, the ophthalmic health management system 120 may obtain allergen data corresponding to the location 108a of the first user 104a.

In some embodiments, one or more of the operations described as being performed in the environment 100 may be performed by an artificial intelligence (AI) model. In some embodiments, the ophthalmic health management system 120 may include an AI model configured to perform the operations described with reference to FIGS. 1A-1C as being performed by the ophthalmic health management system 120. In some embodiments, an AI model may be included on the user devices 106 (e.g., such as in the ophthalmic health management module) and/or a separate device that may be accessed by the user devices 106. In some embodiments, the AI model may include machine learning models such as supervised learning models, unsupervised learning models, deep learning models, neural networks, decision trees, random forests, support vector machines, clustering algorithms, natural language processing models, computer vision models, time series forecasting models, reinforcement learning models, and/or transformer-based models, among others. In these and other embodiments, the AI model utilized by the ophthalmic health management system 120 may be trained using health data (e.g., ophthalmic health data), environmental data (e.g., allergen data), and/or user data to enhance predictive capabilities and/or diagnostic accuracy of the AI model. For example, the training process may involve multiple types of ophthalmic health data that may improve the ability of the AI model to recognize patterns, correlations, and/or predictive indicators related to eye health conditions.

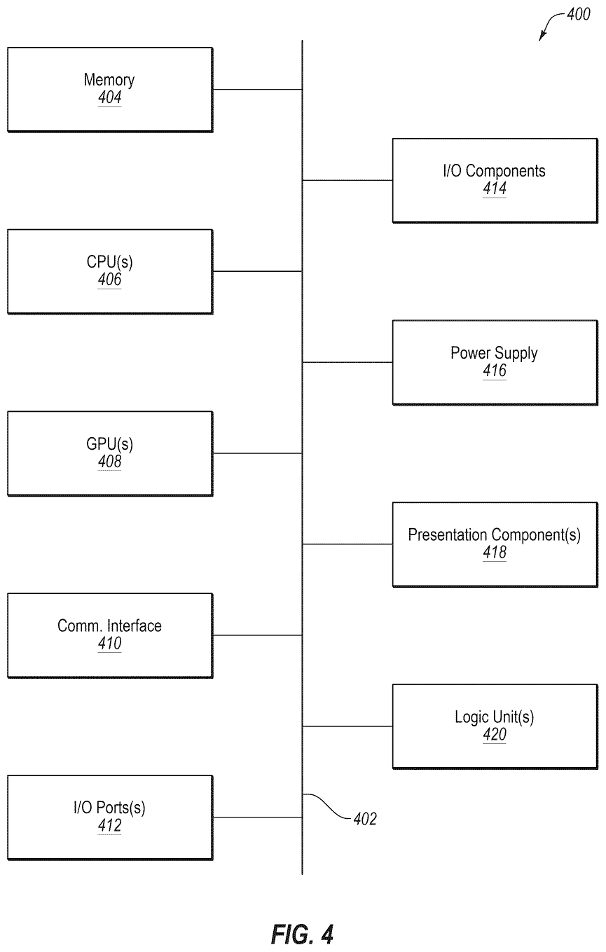

In some embodiments, a separate computing system may include an AI model and the ophthalmic health management system 120 and/or the user devices 106 may direct performance of operations by the AI model on the separate computer system. In the present disclosure, operations described as being performed by the ophthalmic health management system 120 may include operations that the ophthalmic health management system 120 may direct a corresponding computing system (e.g., including an AI model and corresponding computing system) to perform. In these or other embodiments, the ophthalmic health management system 120 may be implemented by one or more computing systems, such as that described in further detail with respect to FIG. 4 of the present disclosure. The ophthalmic health management system 120 is described in further detail with respect to FIGS. 1B and 1C.

FIG. 1B illustrates an example patient side 112 of the example environment 100 of FIG. 1A. The term “patient side” as used in the present disclosure may refer to a portion of the environment 100 corresponding to the interactions between users 104 that are patients and the ophthalmic health management system 120. While the ophthalmic health management system 120, is depicted as being included in the patient side 112 of the environment 100, it will be appreciated that the patient side 112 of the environment 100 may include only patient-facing features of the ophthalmic health management system 120, in some embodiments. The patient side 112 of the example environment 100 is illustrated and described with respect to the first user 104a. However, it will be appreciated that the environment 100 may include additional patient sides that may include the additional user devices 106n, and/or the patient side 112 may include the additional user devices 106n.

As illustrated in FIG. 1B, the first user device 106a may be configured to provide user data 130 associated with the first user 104a to the ophthalmic health management system 120. The ophthalmic health management system 120 may be configured to provide one or more notifications 150 to the first user device 106a based on the user data 130 and/or environmental data 140 associated with the location 108a of the first user 104a.

In some embodiments, the first user device 106a may obtain user data 130 (e.g., via the ophthalmic health management module that may be included in the first user device 106a). The user data 130 may include all data associated with the first user 104a that may be obtained by the first user device 106a. In some embodiments, the user data 130 may include health data 132. In these and other embodiments, health data 132 may include data accessed through EHR/EMR systems, data accessed via one or more health websites (e.g., the MARLÖ® digital eye care platform from Alcon®), data input by the first user 104a, data obtained from a mobile health application on the first user device 106a (e.g., the MARLÖ® application), data measured and/or recorded by the first user device 106a and/or a different device (e.g., a wearable device connected to the first user device 106a), among other health data 132. In these and other embodiments, health data 132 may include user-behavior data that may impact the health of the first user 104a. In some embodiments, the health data 132 may include data obtained from electronic health records, diagnoses, treatments, prescriptions, healthcare provider notes, imaging, patient lab results, and/or medical product purchases (e.g., over-the-counter purchases), among other health data 132.

In some embodiments, the health data 132 may include information related to the ophthalmic health of the first user 104a. For example, the health data 132 may include ophthalmic information associated with the first user 104a and obtained from ophthalmic prescriptions, ophthalmic product purchases ophthalmic conditions and/or diagnoses, ophthalmic medical history, ophthalmic examination information, ophthalmic imaging and/or diagnostic information, ophthalmic data measured via the first user device 106a (e.g., image data corresponding to the eyes obtained by a camera 110), and/or ophthalmic information input into the first user device 106a by the first user 104a, among other ophthalmic health information. In some embodiments, the health data 132 may include information corresponding to ophthalmic characteristics such as blink rate, eye redness, eye tearing, eye swelling, eyelid swelling, pupil characteristics, tear film characteristics, discharge characteristics, and/or other ophthalmic characteristics.

In some embodiments, the health data 132 may include behavioral data associated with the first user 104a that may impact the ophthalmic health of the first user 104a. In these and other embodiments, the behavioral data may include information associated with screen time, contact lens wearing duration and/or replacement schedule, eye rubbing, prescription adherence data, sleep duration and/or quality, and/or engagement in activities that may exacerbate ophthalmic health such as swimming, among other information. In these and other embodiments, the behavioral data may be determined based on data measured and/or obtained by the user device 106a. In these and other embodiments, the behavioral data may be determined based on data obtained by the camera 110 of the first user device 106a. For example, the first user device 106a may determine when a first user 104a begins wearing contact lenses based on image data obtained by the camera 110 and/or may track the amount of time the screen of the user device 106a is unlocked and/or the first user 104a is looking at the user device 106a as a metric for screen time.

In some embodiments, the user data 130 may include location data 134. In these and other embodiments, the location data 134 may correspond to the location 108a of the first user 104a, which may be determined as previously described with respect to FIG. 1A. In some embodiments, the location data 134 may correspond to a current location of the first user 104a. For example, the location data 134 may determine the current location of the first user 104a based on the location of the first user device 106a and/or based on the first user 104a being associated with a particular location at the current time, among other techniques.

In some embodiments, the location data 134 may correspond to a future location of the first user 104a. For example, the location data 134 may indicate that the first user 104a is travelling to a different location than their current location on a specific date and/or at a specific time. In these and other embodiments, a future location may be determined to correspond to the first user 104a when data obtained and/or stored on the first user device 106a indicates that the first user 104a may be travelling to the future location. For example, the future location of the first user data 104a may be determined based on location data 134 in application data (e.g., data from mobile travel applications such as the United Airlines® mobile application), calendar data, commuting pattern data, location history data, email data, messaging data, notification data, and/or other data that may be obtained from the first user device 106a indicating that the first user 104a may be physically present in a different location. For instance, a plane ticket stored in a digital wallet of the first user device 106a may indicate that the first user 104a is travelling to Fort Worth, Texas in the next week and, as a result, Fort Worth, Texas may be determined to be a future location of the first user 104a, which may be included in the location data 134.

In some embodiments, the location data 134 may correspond to a past location of the first user 104a. For example, the location data 134 may indicate that the first user 104a was in one or more locations based on the locations of the first user device 106a and/or other locations that may be associated with the first user 104a based on data obtained from the first user device 106a.

In some embodiments, the user data 130 may be obtained via sensors and/or hardware included in the first user device 106a and/or a different device. For example, the user data 130 may include information obtained by a GPS, a microphone, an accelerometer, a camera 110, and/or other hardware included in the first user device 106a and/or a different device. In these and other embodiments, the first user device 106a may be configured to monitor the ophthalmic health of the first user 104a via the sensors and/or hardware included in the first user device 106a and/or a different device. In these and other embodiments, the first user device 106a may include an ophthalmic health management module that may be a software module, a hardware module, and/or a combination of software modules and hardware modules that may be configured to direct the sensors and/or hardware included in the first user device 106a to obtain the user data 130.

For example, the health data 132 may be obtained by the camera 110 included in the first user device 106a. In these and other embodiments, the camera 110 may be used to monitor the eyes of the first user 104a for certain symptoms and/or conditions associated with allergies, user behaviors, and/or other ophthalmic issues. For example, the camera 110 may obtain image data corresponding to the eyes of the first user 104a, which may be provided to the ophthalmic health management system 120 for analysis. In some embodiments, the camera 110 may continuously obtain image data corresponding to the eyes of the first user 104a. In some embodiments, the camera 110 may periodically obtain image data corresponding to the eyes of the first user 104a. For example, the camera 110 may obtain image data corresponding to the eyes of the first user 104a based on a predetermined interval, based on user input, and/or based on environmental factors (e.g., allergen levels), among other techniques to periodically obtain image data corresponding to the eyes of the first user 104a. For instance, the first user 104a may manually cause image capture through a user interface of the first user device 106a when experiencing symptoms and/or when prompted by the first user device 106a to provide updated ophthalmic health data.

In some embodiments, the camera 110 may be configured to capture image data based on other health data. For example, the camera 110 may be configured to capture image data at intervals based on medication administration times, allowing for assessment of treatment effectiveness. As another example, the camera 110 may also capture image data before and after the user applies eye drops or other treatments to document changes in ophthalmic characteristics.

In these and other embodiments, ophthalmic characteristics may be determined by based on the user data 130. For example, ophthalmic characteristics such as blink rate, eye redness, eye tearing, eye swelling, eyelid swelling, pupil characteristics, tear film characteristics, and/or discharge characteristics, among other ophthalmic characteristics may be determined based on image data obtained by the camera 110. In some embodiments, the ophthalmic characteristics may be determined by the data analysis system 124 as described in more detail below with respect to the data analysis system 124.

In these and other embodiments, the location data 134 may be obtained via a GPS included in the first user device 106a and/or a different device, among other location measurement techniques. In some embodiments, the location data 134 may be determined based on data obtained by the first user device 106a and/or data stored on the first user device 106a as described previously with respect to FIG. 1A. For example, the location data 134 may be determined based on application data (e.g., data from mobile travel applications such as the United Airlines® mobile application), calendar data, commuting pattern data, location history data, email data, messaging data, notification data, and/or other data that may be obtained from the first user device 106a indicating the location 108a of the first user 104a.

In some embodiments and as illustrated in FIG. 1B, the first user device 106a may be configured to provide the user data 130 obtained by the first user device 106a to the ophthalmic health management system 120. For example, the first user device 106a may be configured to provide the location data 134 corresponding to the location 108a that may be associated with the first user 104a to the ophthalmic health management system 120 and/or may be configured to provide health data 132 that may be associated with the first user 104a to the ophthalmic health management system 120. For instance, the first user device 106a may provide ophthalmic health data and the location 108a of the first user 104a to the ophthalmic health management system 120. In these and other embodiments, the user data 130 may be provided to the ophthalmic health management system 120 via the first network 102a as described with respect to FIG. 1A.

In some embodiments, the user data 130 may be processed, filtered, normalized, and/or otherwise manipulated by the first user device 106a before being provided to the ophthalmic health management system 120. In some embodiments, the user data 130 may be provided to the data analysis system 124 as raw data.

In some embodiments, the first user 104a may have a user account associated with the ophthalmic health management system 120, and the user data 130 may be provided to the ophthalmic health management system 120 and associated with the user account of the first user 104a. In some embodiments, the first user 104a may not have a user account associated with the ophthalmic health management system 120, and the first user 104a may input and/or provide the user data 130 to the ophthalmic health management system 120 via the first user device 106a. In these and other embodiments, the ophthalmic health management system 120 may associate the user data 130 with a particular communication session between the ophthalmic health management system 120 and the first user device 106a via, for example, the first network 102a.

As illustrated in FIG. 1B, the ophthalmic health management system 120 may include a data collection system 122, a data analysis system 124, a treatment system 126, and/or a communication system 128, among other systems. Although described as separate systems, the data collection system 122, the data collection system 122, data analysis system 124, the treatment system 126, and/or the communication system 128 may be implemented as a single system.

In some embodiments, the data collection system 122 may include any suitable system, apparatus, or device, configured to perform one or more data collection operations with respect to the first user 104a in the environment 100. For example, in some embodiments, the data collection system 122 may include code and routines configured to allow a computing system to perform one or more data collection operations. Additionally or alternatively, the data collection system 122 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the data collection system 122 may be implemented using a combination of hardware and software. In some embodiments, at least some of the operations associated with the data collection system 122 may be performed by one or more application programming interfaces (APIs), data scraping tools (e.g., crawlers), and/or other data collection tools.

In some embodiments, the data collection system 122 may be configured to gather information from various sources to support the operations of the ophthalmic health management system 120. In some embodiments, the data collection system 122 may obtain user data 130 from the user devices 106 in the environment 100. For example, the data collection system 122 may obtain the user data 130 including the health data 132 and the location data 134 associated with the first user 104a from the first user device 106a. In these and other embodiments, the data collection system 122 may cause the first user device 106a to perform one or more data collection operations. For example, the data collection system 122 may cause the first user device 106a to obtain health data 132 via the camera 110.

In some embodiments, the data collection system 122 may obtain data from a variety of data sources that may be associated with, correlated with, and/or impact ophthalmic health. For example, the data collection system 122 may obtain allergen data from one or more allergen monitoring websites. In these and other embodiments, the data collection system 122 may obtain data from EHR systems, EMR systems, webpages, databases, and/or other sources that may provide data related to ophthalmic health management. As an example, the data collection system 122 may interface with EHR systems to obtain ophthalmic history, ophthalmic prescription information, and/or previous ophthalmic diagnoses, among other ophthalmic information that may be associated with the first user 104a. In some embodiments, the data collection system 122 may obtain health data corresponding to a user account associated with the first user 104a. For example, the data collection system 122 may interface with a user account of the first user 104a that may be associated with an online health platform (e.g., MARLÖ® from Alcon®) to obtain health information associated with the first user 104a.

In these and other embodiments, the data collection system 122 may obtain environmental data 140 from one or more sources. In some embodiments, environmental data 140 may include information corresponding to physical, chemical, and/or biological conditions external to the first user 104a that may impact ophthalmic health. For example, environmental data 140 may include allergen data, air quality data, meteorological data, and/or environmental event data (e.g., the occurrence of wildfires, volcanic eruptions, and/or dust storms, etc.), among other environmental data 140. In some embodiments, allergen data included in the environmental data 140 may include qualitative metrics of allergens (e.g., very high count, high count, moderate count, low count) and/or quantitative metrics of allergens such as pollen count (e.g., tree pollens, grass pollens, and/or weed pollens, among other pollens), mold spore count, and/or other allergen metrics. In these and other embodiments, the data collection system 122 may collect information relating to current and/or forecasted environmental conditions that may impact ophthalmic health, including humidity levels, wind patterns, and/or atmospheric pressure readings that may influence allergen distribution and/or concentration.

In some embodiments, the environmental data 140 may be collected by the data collection system 122 based on the location 108a of the first user 104a. For example, based on the location data 134, the data collection system 122 may determine the past, current, and/or future locations of the first user 104a and may obtain allergen data corresponding to the determined locations. In some embodiments, the data collection system 122 may be configured to obtain environmental data 140 within 10 miles, 20 miles, 30 miles, 40 miles, 50 miles, 60 miles, 70 miles, 80 miles, 90 miles, 100 miles, 200 miles, 300 miles, 400 miles, 500 miles, and/or or any other range of the location 108a of the first user 104a. In these and other embodiments, the data collection system 122 may adjust the range of environmental data 140 based on environmental conditions such as high wind events, general wind direction, etc.

In some embodiments, the data collection system 122 may interface with the data analysis system 124 such that the collected data may be provided to the data analysis system 124 for analysis. In some embodiments, the data collected by the data collection system 122 may be processed, filtered, normalized, and/or otherwise manipulated before being provided to the data analysis system 124. In some embodiments, the data collected by the data collection system 122 may be provided to the data analysis system 124 as raw data.

In some embodiments, the data analysis system 124 may include any suitable system, apparatus, or device, configured to perform one or more analysis operations with respect to the data collected by the data collection system 122. For example, in some embodiments, the data analysis system 124 may include code and routines configured to allow a computing system to perform one or more data analysis operations. Additionally or alternatively, the data analysis system 124 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the data analysis system 124 may be implemented using a combination of hardware and software. In some embodiments, at least some of the operations associated with the data analysis system 124 may be performed by an AI model.

In some embodiments, the data analysis system 124 may be configured to process and/or analyze the user data 130 and/or environmental data 140 to determine a symptom associated with the first user 104a. In some embodiments, the symptom may be a presently-existing symptom (e.g., a current symptom) of the first user 104a and/or a future symptom based on the user data 130 and/or the environmental data 140.

In some embodiments, the data analysis system 124 may predict a future symptom of the first user 104a based on the health data 132 associated with the first user 104a and/or the environmental data 140 corresponding to the location 108a associated with the first user 104a. For example, the data analysis system 124 may predict future symptoms of the first user 104a based on allergens in the location 108a associated with the first user 104a by analyzing allergen data from the environmental data 140 to predict symptom development. In some embodiments, the future symptom may be based on currently elevated allergen levels associated with the location 108a and/or forecasted allergen levels associated with the location 108a. For example, the future symptom of the first user 104a may be determined by the data analysis system 124 based on the current location of the first user device 106a being associated higher allergen levels. For instance, the data analysis system 124 may determine that the first user 104a may experience watery, red, and/or burning eyes in response to the location 108a showing that the first user 104a is currently in Fort Worth, Texas and the environmental data 140 associated with Fort Worth, Texas indicating that cedar pollen levels exceed a predetermined threshold (e.g., a pollen count (grains/m³) threshold).

As another example, the data analysis system 124 may determine the future symptom of the first user 104a based on a future location associated with the first user 104a, and the environmental data 140 in the future location. For instance, the location 108a of the first user 104a may be a future location determined based on a flight itinerary the next week, and the data analysis system 124 may determine that the first user 104a may experience ophthalmic symptoms based on forecasted allergen levels at their destination.

In some embodiments, the data analysis system 124 may analyze historical patterns of allergen exposure and symptom manifestation for the first user 104a to establish predictive models that may identify likely symptom occurrence based on current and/or projected allergen levels. In these and other embodiments, the data analysis system 124 may consider geographic-specific allergen profiles associated with the location 108a to determine which allergens may be most likely to cause symptoms for users in that particular area. In some embodiments, the data analysis system 124 may correlate seasonal allergen patterns with the presence of the first user 104a in the location 108a to assess symptom risk based on the intersection of user location and environmental allergen conditions. Thus, the data analysis system 124 may be configured to determine an allergy symptom that may be associated with a particular allergen in the environmental data 140 that may be present in the location 108a associated with the first user 104a.

In these and other embodiments, an AI model included in the ophthalmic health management system 120 and/or in a separate device that may be communicatively coupled to the ophthalmic health management system 120 may determine based on the allergen data in the location 108a associated with the first user 104a may cause an allergy symptom. In these and other embodiments, the AI model may be trained using environmental data 140, user data 130 (where legally allowed to use user data 130 and/or in manners consistent with laws regarding uses of user data 130), and/or other data to train the AI model. In these and other embodiments, the AI model may be trained using historical symptom data, environmental data patterns, and/or treatment outcome data to improve the accuracy of symptom predictions and treatment recommendations.

In some embodiments, the AI model may identify patterns and/or correlations between specific environmental conditions and the likelihood of symptom development. In these and other embodiments, the AI model may determine which allergens in the environmental data 140 are likely to cause symptoms in the first user 104a. For example, the AI model may utilize the health information (e.g., the health data 132) of the first user 104a to determine whether the allergens in the environmental data 140 are likely to cause symptoms in the first user 104a. For instance, the AI model may have access to health information associated with the first user 104a (e.g., in a closed environment) such as allergen profiles, diagnosed allergens, or other information, which may allow the AI model to predict symptom development in the first user 104a.

In some embodiments, the data analysis system 124 may be configured to determine a presently-existing symptom (e.g., a current symptom) of the first user 104a based on the user data 130 and/or the environmental data 140. For example, the data analysis system 124 may analyze the health data 132 associated with the first user 104a to determine whether the first user 104a may be currently experiencing eye redness, itching, tearing, swelling (e.g., eye swelling and/or eyelid swelling), irritation, blurred vision, burning sensations, ophthalmic discharge, and/or other ophthalmic symptoms. In some embodiments, the data analysis system 124 may determine the presently-existing symptom based on input from the first user 104a (e.g., self-reported symptoms of the first user 104a input to the first user device 106a), based on the data measured and/or recorded by the first user device 106a (e.g., image data from the camera 110), and/or based on behavioral data, among other health data 132.

In some embodiments, the data analysis system 124 may determine the presently-existing symptom and/or the cause of the presently-existing symptom based on the environmental data 140. For example, the data analysis system 124 may determine that a current symptom may be caused by allergens through correlation analysis between environmental data 140 corresponding to the location 108a associated with the first user 104a and the health data 132 associated with the first user 104a. For instance, an AI model may perform the correlation analysis.

In some embodiments, the data analysis system 124 may analyze temporal patterns in the health data 132 (e.g., symptom development and/or symptom progression) to identify whether the symptom started in a temporal range in which allergen levels in the environmental data 140 were elevated in the location 108a of the first user 104a. For example, the data analysis system 124 may detect that eye redness and/or tearing symptoms documented in the health data 132 may coincide with periods when pollen counts exceeded predetermined thresholds in the location 108a of the first user 104a. In some embodiments, the data analysis system 124 may also evaluate the specific allergens present in the environmental data 140 in the location 108a of the first user 104a and match the specific allergens with known allergen sensitivities documented in the health data 132 of the first user 104a to determine the symptom.

In some embodiments, the data analysis system 124 may process and/or analyze user data 130 measured and/or recorded by the first user device 106a to determine a presently-existing symptom in the first user 104a. For example, the data analysis system 124 may process and/or analyze image data obtained from the camera 110 of the first user device 106a to determine one or more ophthalmic characteristics. In these and other embodiments, image data may include data from still images and/or videos. In some embodiments, the ophthalmic characteristics determined by the data analysis system 124 may include blink rate, eye redness, eye tearing, eye swelling, eyelid swelling, pupil characteristics, tear film characteristics, and/or discharge characteristics, among other ophthalmic characteristics. In some embodiments, the ophthalmic characteristics may be utilized by the data analysis system 124 to determine one or more ophthalmic symptoms.

In these and other embodiments, the data analysis system 124 may analyze the image data to determine eye redness by analyzing blood vessel patterns visible in the sclera of the first user 104a. In some embodiments, the degree of redness may be quantified on a numerical scale that may be tracked over time to assess changes in inflammation levels. In some embodiments, the data analysis system 124 may correlate increased redness measurements with elevated allergen levels in the environmental data 140 to identify symptoms that may be attributable to environmental conditions such as elevated allergen levels.

In these and other embodiments, the data analysis system 124 may determine tear film quality and/or quantity based on the image data captured by the camera 110. For example, the data analysis system 124 may determine tear film break-up time by monitoring the appearance of dry spots on the corneal surface following blink events. In some embodiments, the data analysis system 124 may differentiate between non-allergic conditions such as dry eye syndrome and allergic conditions such as allergic conjunctivitis by analyzing tear film characteristics in conjunction with allergen data in the environmental data 140.

In these and other embodiments, the data analysis system 124 may monitor pupil characteristics such as pupil size and/or reactivity through analysis of the image data obtained via the camera 110 to determine pupillary responses to light changes. For example, the data analysis system 124 may determine pupil constriction speed and extent to identify abnormal responses that may indicate neurological conditions or medication effects. In these and other embodiments, the data analysis system 124 may utilize pupillary response data to differentiate between various causes of eye discomfort beyond allergic reactions to allergens in the environmental data 140.

In these and other embodiments, the data analysis system 124 may track blinking rate and/or blinking completeness by analyzing sequential image frames captured by the camera 110. For example, the data analysis system 124 may count blinks per minute and assess whether each blink fully closes the eye or represents a partial blink. In these and other embodiments, the data analysis system 124 may correlate reduced blink rates with digital eye strain conditions and/or may correlate increased blink rates with allergen-induced irritation.

In these and other embodiments, the data analysis system 124 may quantify eyelid swelling by comparing eyelid thickness determined based on the image data received from the camera 110 with baseline measurements associated with the first user 104a. In some embodiments, the data analysis system 124 may detect that changes in eyelid appearance exceeding a predetermined threshold may indicate allergic reactions to allergens in the environmental data 140. In these and other embodiments, the data analysis system 124 may correlate eyelid swelling measurements with specific allergen types present in the environmental data 140.

In these and other embodiments, the data analysis system 124 may quantify eye swelling by comparing measurements of anatomical landmarks in the eye region determined based on the image data received from the camera 110 with baseline measurements associated with the first user 104a. In some embodiments, the data analysis system 124 may detect that changes in the spatial relationship between anatomical landmarks in the eye region exceeding a predetermined threshold may indicate allergic reactions to allergens in the environmental data 140. In these and other embodiments, the data analysis system 124 may correlate eye swelling measurements determined using anatomical landmarks with specific allergen types present in the environmental data 140.

In some embodiments, the data analysis system 124 may analyze discharge patterns and/or tearing by detecting moisture around the eye of the first user 104a area in the image data obtained via the camera 110. In some embodiments, the data analysis system 124 may determine whether the discharge and/or tearing is associated with allergies based on the color, opacity, and/or viscosity of the discharge and/or tearing. For example, clear, watery discharge may be associated with allergies while thick, opaque discharge may be associated with a bacterial and/or viral infection.

In some embodiments, the data analysis system 124 may determine one or more symptoms based on user behavior. For example, the data analysis system 124 may determine a symptom based on user behavior through analysis of behavioral patterns that may indicate ophthalmic conditions and/or based on behavioral patterns that may contribute to ophthalmic conditions. In these and other embodiments, the data analysis system 124 may determine a frequency in the first user 104a rubbing their eyes through the hands of the first user 104a being present in the image data captured by the camera 110. In these and other embodiments, the data analysis system 124 may associate the first user 104a repeatedly bringing their hands to their eye region with eye irritation, eye itching, and/or eye discomfort. In some embodiments, the frequency and/or duration of eye rubbing may be quantified and tracked over time to establish baseline patterns for the first user 104a. In these and other embodiments, in response to the eye rubbing frequency exceeding a predetermined threshold (e.g., as determined by a baseline of the first user 104a), the data analysis system 124 may determine that the first user 104a may be experiencing symptoms such as itching, irritation, and/or general eye discomfort. In some embodiments, the data analysis system 124 may correlate increased eye rubbing behavior with elevated allergen levels in the environmental data 140 to determine whether the symptom may be related to allergic reactions.

In some embodiments, the data analysis system 124 may process image data obtained from the camera 110 to detect features indicative of a contact lens present on the eye of the first user 104a. In these and other embodiments, the data analysis system 124 may utilize the image data obtained from the camera 110 and/or user input data to determine a contact lens wearing duration. For example, the data analysis system 124 may be trained to distinguish between a newly inserted contact lens and a worn contact lens based on the image data obtained from the camera 110. For instance, the data analysis system 124 may analyze the amount of debris, deposit on the lens, tear film on the lens, and/or other lens metrics to distinguish between a newly inserted lens and a worn contact lens. In these and other embodiments, the data analysis system 124 may analyze images over time to determine whether the first user 104a has replaced the contact lens. In these and other embodiments, the contact lens duration may be used to recommend replacing lenses and/or removing lenses. In some embodiments, the data analysis system 124 may evaluate the image data to detect irregularities or artifacts on the surface of the contact lens that may correspond to foreign material or debris, which may indicate that the lens may require cleaning.

In some embodiments, the data analysis system 124 may determine symptoms based on screen time behavior monitored through the first user device 106a. In these and other embodiments, the data analysis system 124 may determine the duration and frequency of screen usage by analyzing application usage data, screen activation patterns, and/or device interaction metrics collected by the first user device 106a. For example, extended screen time periods may be associated with digital eye strain symptoms, which may manifest as dry eyes, blurred vision, eye fatigue, and/or headaches. In some embodiments, the data analysis system 124 may establish personalized screen time thresholds for the first user 104a based on their historical usage patterns and correlate deviations from normal usage with potential symptom development. In some embodiments, in response to screen time exceeding a predetermined screen time threshold, the data analysis system 124 may determine that the first user 104a may be at risk for developing digital eye strain symptoms. In some embodiments, the data analysis system 124 may analyze screen brightness levels, viewing distances, and/or blink rate patterns during screen use to assess the likelihood of symptom occurrence. In some embodiments, the data analysis system 124 may differentiate between screen-related symptoms and allergen-related symptoms by analyzing the temporal relationship between screen usage patterns and environmental allergen levels in the location 108a of the first user 104a.

In some embodiments, the data analysis system 124 may utilize medical product purchase data in determining and/or validating the symptom. For example, the data analysis system 124 may determine that first user 104a has previously purchased ophthalmic care products that may be correlated with specific ophthalmic symptoms. In these and other embodiments, the data analysis system 124 may correlate the timing of product purchases in relation to environmental allergen levels to identify patterns that may indicate symptom occurrence. For example, the data analysis system 124 may determine that the first user 104a frequently purchases ophthalmic products during periods when pollen counts exceed predetermined thresholds in the location 108a associated with the first user 104a.

In some embodiments, the data analysis system 124 may generate a diagnosis 158 based on the symptoms (e.g., current and/or future symptoms) determined by the data analysis system 124. In some embodiments, the data analysis system 124 may correlate specific symptom patterns, manifestations, and/or combinations with known ophthalmic conditions to establish the diagnosis 158. For example, the data analysis system 124 may determine that a combination of eye redness, tearing, and eyelid swelling occurring during periods of elevated pollen counts may indicate seasonal allergic conjunctivitis as the diagnosis 158. As another example, the data analysis system 124 may determine that a combination of blurred vision and increased blink rate occurring during periods of prolonged screen exposure may indicate digital eye strain as the diagnosis 158. In some embodiments, the diagnosis 158 may be generated through pattern recognition algorithms that may compare the determined symptoms against established diagnostic criteria for various ophthalmic conditions. In some embodiments, the data analysis system 124 may utilize evidence-based clinical decision platforms, medical knowledge databases, and/or other clinical support tools in determining the diagnosis 158.

In some embodiments, the data analysis system 124 may correlate the diagnosis 158 with allergens in the environmental data 140 and/or the data analysis system 124 may correlate the diagnosis 158 with user behavior. In some embodiments, the data analysis system 124 may not correlate the diagnosis 158 with allergens in the environmental data 140. In some embodiments, the diagnosis 158 may be validated by a healthcare provider. For example, the diagnosis 158 may be validated by the second user 104b as described in more detail with reference to FIG. 1C.

In some embodiments, the data analysis system 124 may select educational material 156 based on the environmental data 140, the user data 130, the determined symptoms, and/or the diagnosis 158. For example, the data analysis system 124 may correlate specific allergen types present in the environmental data 140 with educational material 156 that may address the particular allergens affecting the first user 104a. For instance, in response to the environmental data 140 indicating elevated tree pollen levels in the location 108a associated with the first user 104a, the data analysis system 124 may identify educational material 156 that may explain the relationship between tree pollen exposure and ophthalmic symptoms. In some embodiments, the educational material 156 may be directed to the relationship between user behavior and ophthalmic symptoms, the relationship between allergens and ophthalmic symptoms, allergen avoidance strategies, and/or treatment options, among other educational material 156. In these and other embodiments, the educational material 156 may be obtained from various sources including healthcare databases, educational institutions, medical organizations, professional associations, and/or online health platforms, among other sources. In some embodiments, the educational material 156 may be provided by and/or associated with an e-commerce platform that may provide purchasing opportunities of products associated with the determined symptoms.

In some embodiments and as described previously, the operations associated with the data analysis system 124 may be performed by an AI model. For example, the data analysis system 124 may include supervised learning models trained on health data 132 and environmental data 140 to recognize symptom patterns associated with allergic responses and/or symptom patterns associated with non-allergic conditions. In some embodiments, the data analysis system 124 may utilize user feedback and/or treatment response data to update the AI model.

In some embodiments, the data analysis system 124 may interface with the treatment system 126 such that the symptoms determined by the data analysis system 124 may be provided to the treatment system 126 to determine an action based on the determined symptoms. In some embodiments, the treatment system 126 may include any suitable system, apparatus, or device, configured to perform one or more analysis operations with respect to the data provided to the treatment system 126. For example, in some embodiments, the treatment system 126 may include code and routines configured to allow a computing system to perform one or more operations associated with determining a treatment. Additionally or alternatively, the treatment system 126 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the treatment system 126 may be implemented using a combination of hardware and software. In some embodiments, at least some of the operations associated with the treatment system 126 may be performed by an AI model.

In some embodiments, the treatment system 126 may be configured to determine an action in response to the determined symptoms. In these and other embodiments, the action may include any preventative, therapeutic, curative, educative, palliative, and/or diagnostic measure that may be determined in response to the symptoms determined by the data analysis system 124, in response to receiving the environmental data 140, and/or in response to receiving the user data 130. In these and other embodiments, the action may include selecting one or more products associated with the symptoms, generating a product order 160 for the one or more products, generating a treatment plan 152, selecting educational material 156, generating the diagnosis 158, and/or sending one or more reminders 162 to the first user device 106a.

In some embodiments, the treatment system 126 may generate the diagnosis 158 in a similar manner as described with respect to the data analysis system 124 or the treatment system 126 may receive the diagnosis 158 from the data analysis system 124. In some embodiments, the treatment system 126 may be configured to generate a treatment plan 152 based on the symptoms and/or diagnosis 158. In these and other embodiments, the treatment system 126 may utilize established clinical protocols, evidence-based treatment guidelines, and/or patient-specific factors to determine the treatment plan 152. For example, the treatment plan 152 determined by the treatment system 126 may be based on the health data 132 associated with the first user 104a.

In some embodiments, the treatment plan 152 may include product recommendations 154, behavioral modifications, environmental adjustments, and/or follow-up care protocols, among other treatment options. For example, in response to the data analysis system 124 identifying symptoms consistent with seasonal allergic conjunctivitis, the treatment system 126 may generate a treatment plan 152 that includes specific antihistamine eye drop recommendations (e.g., Pataday® from Alcon®), allergen avoidance strategies, and/or timing recommendations for administering the antihistamine eye drops based on local pollen forecasts. As another example, in response to the data analysis system 124 identifying symptoms consistent with dry eye syndrome, the treatment system 126 may generate a treatment plan 152 that may include specific lubricant eye drop recommendations (e.g., Systane® from Alcon®), environmental modification strategies, and/or timing recommendations for administering the lubricant eye drops eye drops based on local humidity and/or air quality forecasts. In some embodiments, the treatment system 126 may also incorporate patient-specific factors such as contact lens wear, previous treatment responses, and/or concurrent medications when developing the treatment plan 152. For example, in response to the first user 104a being associated with a contact lens prescription, the treatment plan 152 may further include specific lens cleaning solutions (e.g., OPTI-FREE® and/or CLEAR-CARE products provided by Alcon®), and/or a specific lens cleaning regimen based on the allergen levels in the environmental data 140. In some embodiments, the treatment plan 152 may be validated by a healthcare provider such as the second user 104b as described in more detail with reference to FIG. 1B.

In some embodiments, the treatment system 126 may generate product recommendations 154 based on the symptoms, diagnosis 158, user data 130, and/or environmental data 140. In these and other embodiments, the product recommendations 154 may be based on current symptoms and/or future symptoms. In some embodiments, the treatment system 126 may generate the product recommendations 154 based on actual and/or predicted symptom severity as determined by the data analysis system 124. For example, the data analysis system 124 may predict more severe symptoms for higher grass pollen counts, and the treatment system 126 may recommend stronger and/or more concentrated products based on the severity of the symptoms. In some embodiments, the treatment system 126 may consider patient-specific factors such as the first user's 104a previous product purchases, brand preferences, and/or any documented sensitivities or allergies to ingredients when generating the product recommendations 154, among other considerations.

The treatment system 126 may generate a product order 160 based on the user data 130, the environmental data 140, the determined symptoms, the diagnosis 158, the treatment plan 152, and/or the product recommendations 154. For example, when moderate to very high allergen levels are detected and/or forecasted in the location 108a associated with the first user 104a and the data analysis system 124 determines current and/or future symptom development, the treatment system 126 may generate a product order 160 for appropriate allergy relief products. For instance, the treatment system 126 may generate a product order 160 based on the product recommendations 154.

In some embodiments, the treatment system 126 may place the product order 160 in response to the data analysis system 124 determining the symptoms and/or the diagnosis. In some embodiments, the treatment system 126 may notify the first user 104a to place the product order 160. For example, the treatment system 126 may place the product recommendations 154 in a digital shopping cart of an e-commerce platform and may notify the first user 104a to approve the product order 160. In these and other embodiments, the product order 160 may need the first user 104a to input information to complete the product order 160. For example, billing information may be omitted from the product order 160, and/or the first user 104a may confirm other aspects of information included in the product order 160 such as shipping information. In some embodiments, the treatment system 126 may place the product order 160 based on the user data 130 and/or the environmental data 140 without user input.

In some embodiments, the data analysis system 124 and/or the treatment system 126 may track treatment effectiveness by monitoring changes in symptoms following treatment implementation. In some embodiments, the tracking of treatment effectiveness may include analyzing health data 132 post-treatment (e.g., data obtained from the camera 110, reports from the first user 104a, and/or user behavior data after the treatment plan 152 has been put into effect) to determine the efficacy of the treatment plan 152 generated by the treatment system 126. For example, the data analysis system 124 may monitor reductions in eye redness, decreased tearing, changes in user behavior, and/or user reported comfort levels following the use of recommended eye drops. In these and other embodiments, the data analysis system 124 may quantify these improvements using standardized metrics and may generate reports on treatment effectiveness for patients and/or healthcare providers.

In some embodiments, the treatment system 126 may adapt the treatment plan 152 based on changes in symptoms as detected by the data analysis system 124. In these and other embodiments, in response to symptom pattern changes and/or new symptoms, the treatment system 126 may modify existing treatment plans and/or generate new treatment plans. For example, in response to the data analysis system 124 detecting that allergic symptoms are worsening despite current treatment, the treatment system 126 may recommend stronger medications, additional preventive measures, and/or consultation with healthcare providers, among other recommendations. In some embodiments, the treatment system 126 may adjust the treatment plan 152 based on changes in environmental conditions as reflected in the environmental data 140. For example, in response to allergen levels increasing in the location 108a associated with the first user 104a, the treatment system 126 may recommend different products, may adjust the frequency of product usage, and/or may otherwise adjust the treatment plan 152. For instance, during periods of high pollen counts, the treatment system 126 may recommend an increased frequency of antihistamine eye drop usage.

In some embodiments, the data analysis system 124 and/or the treatment system 126 may monitor patient compliance with the treatment plan 152. In these and other embodiments, monitoring patient compliance may include tracking medication usage patterns, product purchase behaviors, adherence to behavioral recommendations, and/or other behaviors of the first user 104a. For example, the treatment system 126 may monitor whether the first user 104a is using prescribed eye drops according to the recommended schedule by analyzing purchase frequency, usage reports, and/or symptom progression patterns in the user data 130.

As an example, the data analysis system 124 may obtain treatment information from the treatment system 126 and may identify patterns in patient responses to treatments by analyzing treatment effectiveness data over time. In some embodiments, the data analysis system 124 may track changes in ophthalmic characteristics following medication administration and/or product usage. In some embodiments, the data analysis system 124 may correlate symptom improvements with specific treatment interventions to assess treatment efficacy. In some embodiments, the data analysis system 124 may analyze compliance patterns by monitoring whether symptom changes occur at expected intervals following prescribed treatment schedules.

In some embodiments, the communication system 128 may include any suitable system, apparatus, or device, configured to perform one or more communication operations. For example, in some embodiments, the communication system 128 may include code and routines configured to allow a computing system to perform one or more communication operations. Additionally or alternatively, the communication system 128 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the communication system 128 may be implemented using a combination of hardware and software.

In some embodiments, operations associated with the communication system 128 may be performed by an AI model. In some embodiments, the communication system 128 may include a telemedicine component and may facilitate audio and/or video communications between the first user device 106a and the second user device 106b.

In some embodiments, the communication system 128 may be communicatively coupled with the first user device 106a (e.g., via the first network 102a) and may provide one or more notifications 150 to the first user device 106a. In some embodiments, the notifications 150 may be based on the actions determined by the ophthalmic health management system 120 based on the user data 130 and/or the environmental data 140. For example, the notifications 150 may be based on allergens being at a moderate to very-high level (e.g., according to an allergen monitoring website) in the location 108a associated with the first user 104a. In these and other embodiments, the notifications 150 may include the treatment plan 152, the product recommendations 154, the educational material 156, the diagnosis 158, the product order 160, and/or one or more reminders 162, among other aspects that may be provided in the notifications 150. For example, the notifications 150 may provide, based on allergen data in the environmental data 140, the first user 104a with the treatment plan 152, the product recommendations 154, educational material 156, the diagnosis 158 of the first user 104a, the product order 160 (e.g., a notification that the product order 160 has been placed and/or for confirmation to place the product order 160), and/or one or more reminders 162.

In these and other embodiments, the reminders 162 may be based on the treatment plan 152 and may remind the first user 104a to follow the recommended treatment plan 152 (e.g., the medication schedule), to modify their behaviors, and/or to remind them of forecasted allergen events. In these and other embodiments, the notifications 150 may be provided to the first user device 106a based on the treatment plan 152, based on environmental events (e.g., periods of high allergen forecasts in the location 108a associated with the user), and/or based on input from the first user 104a.

In some embodiments, the communication system 128 may be configured to deliver notifications 150 through multiple channels and/or formats. For example, the notifications 150 may include email messages, text messages, automated phone calls, and/or application push notifications, among other notifications 150. In these and other embodiments, the channels and/or formats of the notifications 150 may be adjusted by the first user 104a.

Thus, the patient side 112 of the environment 100 may utilize environmental data 140 to determine current and/or future symptoms of the first user 104a and may provide notifications 150 to the first user 104a that may allow the first user 104a to take one or more preventative and/or corrective measures to address their current and/or future symptoms. For example, the patient side 112 may utilize allergen data based on locations 108a associated with the first user 104a to determine whether the first user 104a may currently be experiencing ophthalmic symptoms from the allergens and/or may experience ophthalmic symptoms from the allergens in the future. In response, the ophthalmic health management system 120 may provide the first user 104a with a customized notification 150 to address their current and/or future ophthalmic symptoms.

As a result, the patient side 112 may enable the first user 104a to help prevent or reduce the severity of ophthalmic symptoms, to determine the actual cause of their ophthalmic symptoms (e.g., whether or not associated with environmental conditions such as allergens), to obtain the appropriate products based on their symptoms, and/or to modify their behavior in response to their current and/or future symptoms. Thus, the patient side 112 of the environment 100 may improve treatment plans, symptom management, compliance with treatment plans, patient education, and/or patient outcomes.

Furthermore, the patient side 112 of the environment may generate product orders 160 based on the user data 130 and/or the environmental data 140 such that the first user 104a may obtain products associated with current and/or future symptoms before the first user 104a experiences the symptoms and/or before the symptoms worsen. As a result, patient outcomes and symptom management may be improved.

FIG. 1C illustrates an example provider side 114 of the example environment 100 of FIG. 1A. The term “provider side” as used in the present disclosure may refer to a portion of the environment 100 corresponding to the interactions between users 104 that are healthcare providers and the ophthalmic health management system 120. While the ophthalmic health management system 120, is depicted as being included in the provider side 114 of the environment 100, it will be appreciated that the provider side 114 of the environment 100 may include only provider-facing features of the ophthalmic health management system 120 in some embodiments. The provider side 114 of the example environment 100 is illustrated and described with respect to the second user 104b. However, it will be appreciated that the environment 100 may include additional provider sides that may include the additional user devices, and/or the provider side 114 may include the additional user devices.

As illustrated in FIG. 1C, the ophthalmic health management system 120 may be configured to provide clinical inputs 170 to the second user device 106b. For example, the ophthalmic health management system 120 may communicate the clinical inputs 170 to the second user device 106b via the second network 102b. The clinical inputs 170 may include any information which a healthcare provider such as the second user 104b may utilize in making a clinical decision regarding a patient such as the first user 104a. In some embodiments, the clinical inputs 170 may include at least a portion of the data collected by the data collection system 122. For example, the clinical inputs 170 may include the user data 130 (e.g., the health data 132 and/or the location data 134) and/or the environmental data 140 (e.g., allergen data).

In some embodiments, the clinical inputs 170 may include one or more determined actions 172. In some embodiments, the determined actions 172 may include any preventative, therapeutic, curative, educative, palliative, and/or diagnostic measure that may be generated by the ophthalmic health management system 120 based on the user data 130 and/or the environmental data 140. For example, the determined actions 172 may be based on the health data 132, the location data 134, allergen data in the environmental data 140, and/or the symptoms identified by the data analysis system 124. In these and other embodiments, the determined actions 172 may include any and/or all of the elements of the notifications 150 described with respect to FIG. 1B. For example, the determined actions 172 may include the treatment plan 152, the product recommendations 154, the educational material 156, the diagnosis 158, the product order 160, and/or the reminders 162 that may be determined based on the symptoms of the first user 104a. In some embodiments, the ophthalmic health management system 120 may communicate (e.g., via the second network 102b) the clinical inputs 170 to the second user device 106b.

In these and other embodiments, the second user 104b may accept the determined actions 172, may modify the determined actions 172, and/or may reject the determined actions 172. For example, the second user 104b may accept the diagnosis 158 is correct but may reject and/or modify the educational material 156 to be sent to the patient. As another example, the second user 104b may select different products for the first user 104a, and the products selected by the second user 104b may replace the previous product recommendations 154 generated by the ophthalmic health management system 120.

Thus, the second user 104b (e.g., a healthcare provider) may, via the second user device 106b, generate validated actions 184 based on the clinical inputs 170 provided to the second user device 106b. For example, the validated actions 184 may include a validated treatment plan, validated product recommendations, validated educational material, a validated diagnosis, a validated product order, and/or validated reminders. In these and other embodiments, the validated actions 184 may include approved determined actions, modified determined actions, and/or actions generated by the second user 104b. In some embodiments, the notifications 150 may be based on the validated actions 184 that the ophthalmic health management system 120 may receive.

In these and other embodiments, the second user 104b may provide clinical outputs 180 via the second user device 106b to the ophthalmic health management system 120. In some embodiments, the clinical output may include the validated actions 184 discussed previously and/or provider data 182. In some embodiments, the provider data 182 may include information generated by healthcare providers prior to receiving and/or after receiving the clinical inputs 170 received from the ophthalmic health management system 120.

In some embodiments, the provider data 182 may include information associated with the first user 104a such as an ophthalmic prescription for the first user 104a (e.g., a contact lens prescription). In some embodiments, the provider data 182 may include clinical notes, diagnostic assessments, treatment recommendations, and/or other health data that may be associated with the first user 104a. In these and other embodiments, the provider data 182 may be incorporated in the health data 132 associated with the first user 104a.

In these and other embodiments, the second user device 106b may provide the clinical outputs 180 to the ophthalmic health management system 120 (e.g., via the second network 102b). In some embodiments, the ophthalmic health management system 120 may utilize the clinical outputs 180 to refine and/or improve the symptom determination and treatment recommendations based on user data 130 and/or environmental data 140. For example, an AI model may utilize the clinical outputs 180 to refine pattern recognition and/or correlation analysis to determine symptoms based on user data 130 and/or environmental data 140.

In these and other embodiments, the ophthalmic health management system 120 may generate the notifications 150 based on the clinical outputs 180. For example, the ophthalmic health management system 120 may provide a notification 150 to the first user device 106a based on the validated actions 184 received from the second user device 106b. As a result, the provider side 114 of the environment 100 may help the ophthalmic health management system 120 to generate personalized and healthcare provider validated notifications 150 to the first user 104a based on their user data 130 and/or environmental data 140 corresponding to the location 108a associated with the first user 104a.

Modifications, additions, or omissions may be made to FIGS. 1A-1C without departing from the scope of the present disclosure. For example, the environment 100 may include more or fewer elements depending on the implementation. For instance, in some embodiments, the environment 100 may not include the provider side 114 illustrated in FIG. 1C, and the ophthalmic health management system 120 may provide the notifications 150 without healthcare provider input. Further, the environment 100 may be configured to perform any number of operations as compared to those explicitly described. In addition, the principles described may be applied to ophthalmic health management beyond allergen management and/or may be applied to other areas of health management that may be impacted by environmental conditions.

As described previously, in some embodiments, the provider side 114 of the environment 100 may be omitted such that the second network 102b, the second user device 106b, and/or the second user 104b may be omitted from the environment 100.

In some embodiments, the ophthalmic health management system 120 may provide notifications 150 based on any of the health data 132, the location data 134, and/or the environmental data 140. In some embodiments, the notifications 150 may include more or less elements than those specifically illustrated and described with respect to FIG. 1B. For example, in some embodiments, the treatment plan 152, the product recommendations 154, the educational material 156, the diagnosis 158, the product order 160, and/or the reminder 162 may be omitted from the notifications 150.

In some embodiments, the clinical inputs 170 and/or clinical outputs 180 may include more or less elements than specifically illustrated and described with respect to FIG. 1C. For example, the clinical inputs 170 may not include the user data 130 and/or the environmental data 140, and the clinical outputs 180 may not include the provider data 182.

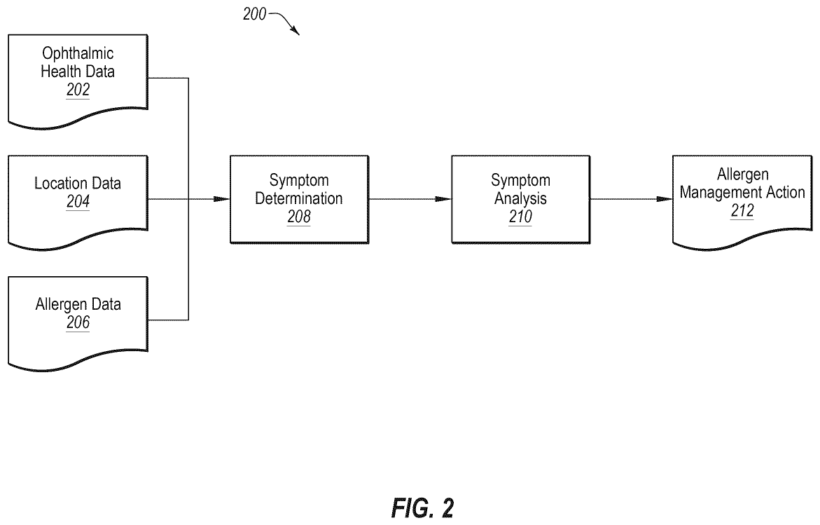

FIG. 2 illustrates an example process 200 that may be performed to manage ophthalmic health in response to allergens, according to one or more embodiments of the present disclosure. Each operation or block of the process 200 described herein, may comprise a computing process that may be performed using any combination of hardware, firmware, and/or software. For instance, various functions may be carried out by a processor executing instructions stored in memory. The process 200 may also be embodied as computer-usable instructions stored on computer storage media. The process 200 may be provided by a standalone application, a service or hosted service (standalone or in combination with another hosted service), as a microservice via an application programming interface (API) or a plug-in to another product, to name a few. In addition, the process 200 is described, by way of example, with respect to the environment 100 of FIG. 1. However, the process 200 may additionally or alternatively be executed in other environments, by any one system or any combination of systems, including, but not limited to, those described herein. Further, to ease explanation, the description of the process 200 is given with respect to managing ophthalmic health in response to allergens, however such a process may be used for other facets of health management and/or with respect to other environmental conditions that may have an impact on ophthalmic health.

In some embodiments, the process 200 may include obtaining ophthalmic health data 202, which may be similar to the health data 132 described with respect to FIGS. 1B and 1C. For example, the first user device 106a may obtain ophthalmic health data 202 associated with the first user 104a. In some embodiments, the ophthalmic health data 202 may include ophthalmic prescription information, ophthalmic product purchase information, ophthalmic information measured and/or recorded by a user device, user reported ophthalmic symptom information, ophthalmic information generated by healthcare providers, contact lens usage information, user behavior information, and/or other information that may be related to the ophthalmic health of a user. For example, the first user device 106a may obtain ophthalmic health data 202 pertaining to one or more of the ophthalmic characteristics described with respect to FIG. 1B.

In some embodiments, the process 200 may include obtaining location data 204, which may be similar to the location data 134 described with respect to FIG. 1B. For example, the location data 204 may correspond to the location 108a associated with the first user 104a. In these and other embodiments, the location data 204 may correspond to a future location and/or a current location of a user. The location data 204 may be obtained in a similar manner as described with respect to FIG. 1B.

In some embodiments, the process 200 may include obtaining allergen data 206, which may be similar to the environmental data 140 described with respect to FIG. 1B and obtained in a similar fashion. For example, the data collection system 122 may obtain allergen data 206 from an allergen monitoring website such as allergy.com® and/or pollen.com®. In some embodiments, the allergen data 206 may include qualitative metrics (e.g., very high count, high count, moderate count, low count) and/or quantitative metrics of allergens such as pollen count (e.g., tree pollens, grass pollens, and/or weed pollens among other pollens), mold spore count, and/or other allergen metrics. In these and other embodiments, the allergen data 206 may include current (e.g., real-time) and/or forecasted allergen data that may impact ophthalmic health, including allergen levels and/or types.

In some embodiments, the qualitative metrics may be based on the quantitative metrics in the allergen data 206. For example, the qualitative metrics may include low, moderate, high, and/or very high allergen counts, and the qualitative metrics may be associated with specific ranges of quantitative metrics for measuring allergen count. For instance, the allergen data 206 may be measured in allergen count per cubic meter of air (e.g., g/m³), and the qualitative metrics may be determined based on whether the allergen data falls within a specific range of allergen count per cubic meter of air. As an example, general threshold ranges for tree pollen may be 1-14 g/m³ (low), 15-89 g/m³ (moderate), 90-1,499 g/m³ (high), and 1,500+ g/m³ (very high), general threshold ranges for weed pollen may be 1-9 g/m³ (low), 10-49 g/m³ (moderate), 50-499 g/m³ (high), and 500+ g/m³ (very high), general threshold ranges for grass pollen may be 1-4 g/m³ (low), 5-19 g/m³ (moderate), 20-199 g/m³ (high), and 200+ g/m³ (very high), and/or general threshold ranges for mold spores may be 1-6,499 g/m³ (low), 6,500-12,999 g/m³ (moderate), 13,000-49,999 g/m³ (high), and 50,000+ g/m³ (very high). In some embodiments, the allergen metrics may be determined by a third-party such as an allergen monitoring website.

In some embodiments, allergen data 206 may be obtained based on the location data 204. For example, the allergen data 206 may correspond to the location 108a associated with the first user 104a. In these and other embodiments, the allergen data 206 may be current and/or forecasted data in the locations associated with the location data 204. In these and other embodiments, forecasted allergen data may include data corresponding to a daily forecast, a weekly forecast, and/or a monthly forecast in the locations associated with the location data 204.

In some embodiments, the process 200 may include a symptom determination operation 208 (hereinafter “symptom determination 208”) configured to determine one or more current and/or future symptoms of a user based on the ophthalmic health data 202, the location data 204, and/or the allergen data 206. For example, the data analysis system 124 may determine one or more symptoms based on the ophthalmic health data 202, the location data 204, and/or the allergen data 206. For instance, the symptom determination 208 may determine that the first user 104a may experience ophthalmic symptoms based on a tree pollen forecast in the moderate to very high ranges of tree pollen. The symptom determination operation 208 may determine the one or more symptoms in a similar manner as described with respect to the data analysis system 124.

In some embodiments, the process 200 may include a symptom analysis operation 210 (hereinafter “symptom analysis 210”). In these and other embodiments, the symptom analysis 210 may analyze the symptoms determined during the symptom determination 208 to generate one or more allergen maintenance actions 212. The symptom analysis 210 may be performed by the data analysis system 124 and/or the treatment system 126 of the ophthalmic health management system 120 as described with respect to FIG. 1B.

In some embodiments, the allergen maintenance actions 212 may include any preventative action, therapeutic action, curative action, educative action, palliative action, diagnostic action, and/or other action configured to prevent, correct, and/or mitigate allergy symptoms. In some embodiments, the allergen maintenance actions 212 may include selecting one or more products associated with the allergy symptoms (e.g., an antihistamine), generating a product order (e.g., an order for antihistamines) based on the allergy symptoms, generating an allergy treatment plan, providing allergen educational material, directing a user to an e-commerce website associated with ophthalmic products configured to treat allergies, and/or generating a diagnosis (e.g., a diagnosis of allergic conjunctivitis) based on the allergy symptoms, among other allergen maintenance actions 212.

In these and other embodiments, a notification may be provided to a user based on the allergen maintenance actions 212. For example, the allergen maintenance actions 212 may be included in the notifications 150 described with respect to FIG. 1B. As an example, a notification 150 may be provided to the first user 104a that may include educational material 156 and/or may direct the first user 104a to a website selling ophthalmic care products configured to treat the symptoms determined in the symptom determination 208.

Thus, the process 200 may determine when a user (e.g., the first user 104a) may be at risk of developing allergy symptoms and/or may be currently experiencing allergy symptoms based on allergen data 206 in their current and/or future locations. The process 200 may then determine one or more allergen management actions 212 configured to help the user manage their current and/or future allergy symptoms in response. As a result, allergy symptoms may be managed more effectively and patient outcomes may be improved.

Modifications, additions, or omissions may be made to FIG. 2 without departing from the scope of the present disclosure. For example, the process 200 may include more or fewer operations depending on the implementation. In some embodiments, the ophthalmic health data 202 may be omitted and the allergen management action 212 may be generated based on the location data 204 and the allergen data 206. In some embodiments, the symptom determination 208 and the symptom analysis 210 may be omitted, and the allergen management action 212 may be generated based solely on the allergen data 206 and the location data 204.

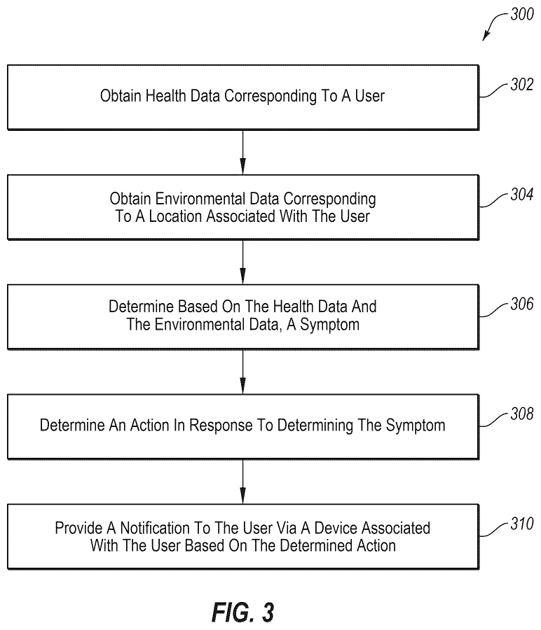

FIG. 3 is a flow diagram illustrating a method 300 of ophthalmic health management. One or more operations of the method 300 may be performed by any suitable system, apparatus, or device such as, for example, the user devices 106 of FIG. 1A, the ophthalmic health management system 120 of FIG. 1A, and/or a computing system such as that described with respect to FIG. 4 of the present disclosure. Furthermore, one or more operations of the method 300 may be performed by an AI model. In addition, the method 300 may be performed as part of the process 200 described with respect to FIG. 2.

At block 302, health data corresponding to a user may be obtained. For example, the health data 132 corresponding to the first user 104a may be obtained as described with respect to FIGS. 1A-1C.

At block 304, environmental data corresponding to a location associated with the user may be obtained. For example, environmental data 140 corresponding to the location 108a associated with the first user 104a may be obtained as described with respect to FIGS. 1A-1C. In some embodiments, the environmental data may include allergen data associated with the location, and the symptom may be an allergy symptom associated with an allergen identified in the allergen data.

At block 306, based on the health data and the environmental data, a symptom may be determined. For example, the ophthalmic health management system 120 may determine a symptom based on the health data 132 and the environmental data 140. In some embodiments, the symptom may be a future symptom.

At block 308, an action may be determined in response to determining the symptom. For example, an action may be determined by the ophthalmic health management system 120 in response to determining the symptom.

In some embodiments, the action may include selecting one or more products associated with the symptom, generating an order for the one or more products based on the symptom, generating a diagnosis based on the symptom, and/or generating a treatment plan based on the symptom.

At block 310, a notification may be provided to the user via a device associated with the user based on the determined action. For example, the notification 150 may be provided to the first user 104a via the first user device 106a based on the determined action.

Modifications, additions, or omissions may be made to the method 300 without departing from the scope of the present disclosure. For example, the operations of method 300 may be implemented in differing order in some instances. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

In some embodiments, the health data may include ophthalmic health data and/or data obtained from the device. In these and other embodiments, the health data may include obtaining at least a portion of the health data based on image data obtained by a camera in the device associated with the user. In these and other embodiments, the method 300 may further include determining the symptom based on the portion of the health data. In these and other embodiments, the portion of the health data may correspond to one or more ophthalmic characteristics including blink rate, eye redness, eye tearing, eye swelling, eyelid swelling, pupil characteristics, tear film characteristics, and/or discharge characteristics.

In some embodiments, the location associated with the user may be a future location determined based on user data that corresponds to the user, and the user data may be obtained from the device.

### EXAMPLE COMPUTING SYSTEM

FIG. 4 is a block diagram of an example computing system 400 suitable for use in implementing some embodiments of the present disclosure. Computing system 400 may include an interconnect system 402 that directly or indirectly couples the following devices: memory 404, one or more central processing units (CPUs) 406, one or more graphics processing units (GPUs) 408, a communication interface 410, I/O ports 412, input/output components 414, a power supply 416, one or more presentation components 418 (e.g., display(s)), and one or more logic units 420.

Although the various blocks of FIG. 4 are illustrated as connected via the interconnect system 402 with lines, this is not intended to be limiting and is for clarity only. For example, in some embodiments, a presentation component 418, such as a display device, may be considered an I/O component 414 (e.g., if the display is a touch screen). As another example, the CPUs 406 and/or GPUs 408 may include memory (e.g., the memory 404 may be representative of a storage device in addition to the memory of the GPUs 408, the CPUs 406, and/or other components). In other words, the computing system of FIG. 4 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 4.

The interconnect system 402 may represent one or more links or busses, such as an address bus, a data bus, a control bus, or a combination thereof. The interconnect system 402 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 406 may be directly connected to the memory 404. Further, the CPU 406 may be directly connected to the GPU 408. Where there is direct, or point-to-point, connection between components, the interconnect system 402 may include a PCIe link to carry out the connection. In these examples, a PCI bus need not be included in the computing system 400.

The memory 404 may include any of a variety of computer-readable media. The computer-readable media may be any available media that may be accessed by the computing system 400. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 404 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 400. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 406 may be configured to execute at least some of the computer-readable instructions to control one or more components of the computing system 400 to perform one or more of the methods and/or processes described herein. The CPU(s) 406 may each include one or more cores (e.g., one, two, four, eight, twenty-eight, seventy-two, etc.) that are capable of handling a multitude of software threads simultaneously. The CPU(s) 406 may include any type of processor, and may include different types of processors depending on the type of computing system 400 implemented (e.g., processors with fewer cores for mobile devices and processors with more cores for servers). For example, depending on the type of computing system 400, the processor may be an Advanced RISC Machines (ARM) processor implemented using Reduced Instruction Set Computing (RISC) or an x86 processor implemented using Complex Instruction Set Computing (CISC). The computing system 400 may include one or more CPUs 406 in addition to one or more microprocessors or supplementary co-processors, such as math co-processors.

In addition to or alternatively from the CPU(s) 406, the GPU(s) 408 may be configured to execute at least some of the computer-readable instructions to control one or more components of the computing system 400 to perform one or more of the methods and/or processes described herein. One or more of the GPU(s) 408 may be an integrated GPU (e.g., with one or more of the CPU(s) 406 and/or one or more of the GPU(s) 408 may be a discrete GPU. In embodiments, one or more of the GPU(s) 408 may be a coprocessor of one or more of the CPU(s) 406. The GPU(s) 408 may be used by the computing system 400 to render graphics (e.g., 3D graphics) or perform general purpose computations. For example, the GPU(s) 408 may be used for General-Purpose computing on GPUs (GPGPU). The GPU(s) 408 may include hundreds or thousands of cores that are capable of handling hundreds or thousands of software threads simultaneously. The GPU(s) 408 may generate pixel data for output images in response to rendering commands (e.g., rendering commands from the CPU(s) 406 received via a host interface). The GPU(s) 408 may include graphics memory, such as display memory, for storing pixel data or any other suitable data, such as GPGPU data. The display memory may be included as part of the memory 404. The GPU(s) 408 may include two or more GPUs operating in parallel (e.g., via a link). The link may directly connect the GPUs (e.g., using NVLINK) or may connect the GPUs through a switch (e.g., using NVSwitch). When combined together, each GPU 408 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 406 and/or the GPU(s) 408, the logic unit(s) 420 may be configured to execute at least some of the computer-readable instructions to control one or more components of the computing system 400 to perform one or more of the methods and/or processes described herein. In embodiments, the CPU(s) 406, the GPU(s) 408, and/or the logic unit(s) 420 may discretely or jointly perform any combination of the methods, processes and/or portions thereof. One or more of the logic units 420 may be part of and/or integrated in one or more of the CPU(s) 406 and/or the GPU(s) 408 and/or one or more of the logic units 420 may be discrete components or otherwise external to the CPU(s) 406 and/or the GPU(s) 408. In embodiments, one or more of the logic units 420 may be a coprocessor of one or more of the CPU(s) 406 and/or one or more of the GPU(s) 408.

Examples of the logic unit(s) 420 include one or more processing cores and/or components thereof, such as Tensor Cores (TCs), Tensor Processing Units(TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 410 may include one or more receivers, transmitters, and/or transceivers that enable the computing system 400 to communicate with other computing systems via an electronic communication network, including wired and/or wireless communications. The communication interface 410 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 412 may enable the computing system 400 to be logically coupled to other devices including the I/O components 414, the presentation component(s) 418, and/or other components, some of which may be built into (e.g., integrated in) the computing system 400. Illustrative I/O components 414 include a microphone, mouse, keyboard, joystick, game pad, game controller, satellite dish, scanner, printer, wireless device, etc. The I/O components 414 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 400. The computing system 400 may include depth cameras, such as stereoscopic camera systems, infrared camera systems, RGB camera systems, touchscreen technology, and combinations of these, for gesture detection and recognition. Additionally, the computing system 400 may include accelerometers or gyroscopes (e.g., as part of an inertia measurement unit (IMU)) that enable detection of motion. In some examples, the output of the accelerometers or gyroscopes may be used by the computing system 400 to render immersive augmented reality or virtual reality.

The power supply 416 may include a hard-wired power supply, a battery power supply, or a combination thereof. The power supply 416 may provide power to the computing system 400 to enable the components of the computing system 400 to operate.

The presentation component(s) 418 may include a display (e.g., a monitor, a touch screen, a television screen, a heads-up-display (HUD), other display types, or a combination thereof), speakers, and/or other presentation components. The presentation component(s) 418 may receive data from other components (e.g., the GPU(s) 408, the CPU(s) 406, etc.), and output the data (e.g., as an image, video, sound, etc.).

Modifications, additions, or omissions may be made to FIG. 4 without departing from the scope of the present disclosure. For example, the computing system 400 may include more or fewer elements depending on the implementation. Further, the computing system 400 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example. The following is a non-limiting summary of some example implementations presented herein.

Example 1. A method of ophthalmic health management comprising:
obtaining health data corresponding to a user;
obtaining environmental data corresponding to a location associated with the user;
determining, based on the health data and the environmental data, a symptom;
determining an action in response to determining the symptom; and
providing a notification to the user via a device associated with the user based on the determined action.

Example 2. The method of Example 1, wherein the environmental data includes allergen data associated with the location, and the symptom is an allergy symptom associated with an allergen identified in the allergen data

Example 3: The method of Examples 1 or 2, wherein the symptom is a future symptom.

Example 4: The method of any of Examples 1-3, wherein the health data includes at least one of: ophthalmic health data or data obtained from the device.

Example 5: The method of any of Examples 1-4, wherein the action includes at least one of:
selecting one or more products associated with the symptom;
generating, based on the symptom, an order for the one or more products; or
generating a treatment plan based on the symptom.

Example 6: The method of any of Examples 1-5, further comprising obtaining at least a portion of the health data based on image data obtained by a camera in the device associated with the user.

Example 7: The method of Example 6, further comprising determining, based on the portion of the health data, the symptom.

Example 8: The method of Example 7, wherein the action includes at least one of:
generating a diagnosis based on the symptom;
selecting one or more products associated with the symptom;
generating, based on the symptom, an order for the one or more products; or
generating a treatment plan based on the symptom.

Example 9: The method of Example 6, wherein the portion of the health data corresponds to one or more ophthalmic characteristics, the ophthalmic characteristics including at least one of:
blink rate;
eye redness;
eye tearing;
eye swelling;
eyelid swelling;
pupil characteristics;
tear film characteristics; or
discharge characteristics.

Example 10: The method of any of Examples 1-9, wherein the location associated with the user is a future location determined based on user data that corresponds to the user, the user data obtained from the device.

Example 11: An ophthalmic health management system comprising a computing system configured to cause performance of the method of any of Examples 1-10.

Example 12: One or more non-transitory computer-readable storage media having instructions stored thereon that, in response to execution by one or more processors, cause performance of the method of any of Examples 1-10.

What is claimed is:

1. A method of ophthalmic health management comprising:
obtaining health data corresponding to a user;
obtaining environmental data corresponding to a location associated with the user;
determining, based on the health data and the environmental data, a symptom;
determining an action in response to determining the symptom; and
providing a notification to the user via a device associated with the user based on the determined action.

2. The method of claim 1, wherein the environmental data includes allergen data associated with the location, and the symptom is an allergy symptom associated with an allergen identified in the allergen data.

3. The method of claim 1, wherein the symptom is a future symptom.

4. The method of claim 1, wherein the health data includes at least one of: ophthalmic health data or data obtained from the device.

5. The method of claim 1, wherein the action includes at least one of:
selecting one or more products associated with the symptom;
generating, based on the symptom, an order for the one or more products; or
generating a treatment plan based on the symptom.

6. The method of claim 1, further comprising obtaining at least a portion of the health data based on image data obtained by a camera in the device associated with the user.

7. The method of claim 6, further comprising determining, based on the portion of the health data, the symptom.

8. The method of claim 7, wherein the action includes at least one of:
generating a diagnosis based on the symptom;
selecting one or more products associated with the symptom;
generating, based on the symptom, an order for the one or more products; or
generating a treatment plan based on the symptom.

9. The method of claim 6, wherein the portion of the health data corresponds to one or more ophthalmic characteristics, the ophthalmic characteristics including at least one of:
blink rate;
eye redness;
eye tearing;
eye swelling;
eyelid swelling;
pupil characteristics;
tear film characteristics; or
discharge characteristics.

10. The method of claim 1, wherein the location associated with the user is a future location determined based on user data that corresponds to the user, the user data obtained from the device.

11. An ophthalmic health management system comprising:
a computing system configured to cause performance of operations, the operations comprising:
obtaining health data corresponding to a user;
obtaining environmental data corresponding to a location associated with the user;
determining, based on at least one of the health data and the environmental data, a symptom;
determining an action in response to determining the symptom; and
providing a notification to the user via a device associated with the user based on the determined action.

12. The ophthalmic health management system of claim 11, wherein the environmental data includes allergen data associated with the location, and the symptom is an allergy symptom associated with an allergen identified in the allergen data.

13. The ophthalmic health management system of claim 11, wherein the symptom is a future symptom.

14. The ophthalmic health management system of claim 11, wherein the health data includes at least one of: ophthalmic health data or data obtained from the device.

15. The ophthalmic health management system of claim 11, wherein the action includes at least one of:
selecting one or more products associated with the symptom;
generating, based on the symptom, an order for the one or more products; or
generating a treatment plan based on the symptom.

16. The ophthalmic health management system of claim 11, wherein the operations further comprise:
obtaining at least a portion of the health data via a camera in the device associated with the user; and
determining, based on the portion of the health data, the symptom of the user.

17. The ophthalmic health management system of claim 16, wherein the action includes at least one of:
generating a diagnosis based on the symptom;
selecting one or more products associated with the symptom;
generating, based on the symptom, an order for the one or more products; or
generating a treatment plan based on the symptom.

18. The ophthalmic health management system of claim 16, wherein the portion of the health data corresponds to one or more ophthalmic characteristics, the ophthalmic characteristics including at least one of:
blink rate;
eye redness;
eye tearing;
eye swelling;
eyelid swelling;
pupil characteristics;
tear film characteristics; or
discharge characteristics.

19. The ophthalmic health management system of claim 11, wherein the location associated with the user is a future location determined based on user data that corresponds to the user, the user data obtained from the device.

20. One or more non-transitory computer-readable storage media having instructions stored thereon that, in response to execution by one or more processors, cause performance of operations, the operations comprising:
obtaining health data corresponding to a user;
obtaining environmental data corresponding to a location associated with the user;
determining, based on at least one of the health data and the environmental data, a symptom;
determining an action in response to determining the symptom; and
providing a notification to the user via a device associated with the user based on the determined action.
